(19) United States
(12) Patent Application Publication
(10) Pub. No.: US 2021/0198636 A1
(43) Pub. Date: Jul. 1, 2021

(54) Use of Syncytin for Targeting Drug and Gene Delivery to Lung Tissue

(71) Applicants: Genethon, Evry (FR); Institut National de la Santé et de la Recherche Médicale, Paris (FR); Université d'Evry val d'Essonne, Evry (FR)

(72) Inventors: Anne Galy, Fontainebleau (FR); Youna Coquin, Alfortville (FR); Maxime Ferrand, Mennecy (FR)

(73) Assignees: Genethon, Evry (FR); Institut National de la Santé et de la Recherche Médicale, Paris (FR); Université d'Evry val d'Essonne, Evry (FR)

(21) Appl. No.: 16/757,590

(22) PCT Filed: Oct. 19, 2018
(86) PCT No.: PCT/EP2018/078809

§ 371 Date: Apr. 20, 2020

(30) Foreign Application Priority Data
Oct. 20, 2017 (EP) 17306447.8

(51) Int. Cl. C12N 7/04; A61P 11/00; C12N 15/113; A61K 48/00

(52) U.S. Cl. CPC C12N 7/04; A61P 11/00; C12N 2740/15023; A61K 48/0091; C12N 15/113

(57) ABSTRACT

The invention relates to a pharmaceutical composition for targeting drug delivery including gene delivery to lung tissue, comprising at least a therapeutic drug or gene associated to a syncytin protein, and its use in the prevention and/or treatment of lung diseases, in particular in gene therapy of said diseases using lentiviral vector particles or lentivirus-like particles pseudotyped with syncytin protein.

### FIELD OF THE INVENTION

The present invention relates to pharmaceutical compositions for targeting lung tissue and to their use in the prevention and/or treatment of lung diseases. More particularly, the present invention relates to the use of syncytin for targeting drug delivery including gene delivery to the lungs.

### BACKGROUND OF THE INVENTION

Targeted therapy holds promise for the treatment of lung diseases such as notably cystic fibrosis. However, the clinical relevance of targeted drug delivery including gene delivery lies in the ability to specifically target a drug or a drug carrier to minimize drug-originated systemic toxic effects, and in particular to minimize liver toxicity.

Plasmids, adenovirus-, adeno-associated virus-, and retrovirus-based vectors have been developed for use in targeted therapy, but these have been limited by poor efficiency of gene transfer, host immune responses directed against the viral vector, or the requirement for proliferating cells for transduction.

The tropism of naturally-occurring viruses is due to one or several molecular determinants that confer a specific ability to bind to cells and to enter into these cells to infect them. The molecular determinants of virus tropism may be exploited to generate new tools for gene delivery. In the case of enveloped viruses such as retroviruses this is called pseudotyping, commonly used with retroviral or lentiviral (typically HIV-1 based) vectors, by anchoring exogenous proteins into their lipid envelopes. Pseudotypes are generally capable of binding to cellular receptors and fuse the vector membrane with the target cell membrane to enable entry.

Lentiviral vectors (LV) which are enveloped RNA particles measuring approximately 120 nm in size are efficient drug delivery tools and more particularly gene delivery tools. The LV binds to, and enters into target cells through its envelope proteins which confer its pseudotype. Once the LV has entered into the cells, it releases its capsid components and undergoes reverse transcription of the lentiviral RNA before integrating the proviral DNA into the genome of target cells. Non-integrative lentiviral vectors have been generated by modifying the properties of the vector integration machinery and can be used for transient gene expression. Virus-like particles lacking a provirus have also been generated and can be used to deliver proteins or messenger RNA. LV can be used for example, for gene addition, RNA interference, exon skipping or gene editing. All of these approaches can be facilitated by tissue or cell targeting of the LV via its pseudotype.

A common pseudotyping protein is vesicular stomatitis virus glycoprotein (VSVg), which is known for conferring broad tropism, particle stability and high titer. However, in vivo VSVg binds complement, and when used in vivo, targets transgene delivery to the liver and is immunogenic (Ciré et al. Plos One, 9, e101644, 2014). New pseudotypes are therefore needed for in vivo gene delivery.

There is thus a need for providing composition comprising at least a drug for use in the prevention and/or treatment of the lung diseases, which targets lung tissue and which has no liver toxicity (i.e. which does not target the liver).

In particular, it would be highly relevant to provide a stable virus or viral vector, for improving delivery of a drug, notably a gene, into lung tissues. Such a virus or viral vector would have to be fully-tolerated, specific for lung tissue, and adequate for systemic administration.

Syncytin, are endogenous retroviral virus (ERV syncytins) envelope glycoproteins which have fusogenic properties (Dupressoir et al., Proceedings of the National Academy of Sciences of the United States of America, 2005, 102, 725-730; Lavialle et al., Phil. Trans. R. Soc. B., 2013, 368:20120507). Human endogenous retroviral envelope glycoprotein encoded by the ERVW-1 gene (ENSG00000242950; also known as syncytin-1 or HERV-W) has been described for its fusogenic properties in patent application EP2385058. Said application describes its use in cancer treatment, by the formation of syncytia. Murine syncytins encompasse murine syncytin-A (i.e.: mus musculus syncytin-A, synA) and murine syncytin-B (i.e.: mus musculus syncytin-B, synB).

However, Ly6e the candidate receptor for Syncytin A has been shown to be rather ubiquitously expressed in mouse adult tissues (Bacquin et al., J. Virol. Doi:10.1128/JVI.00832-17, published only 5 Jul. 2017). More particularly, transcript levels of Ly6e in the mouse liver were found to be at more than 60% of maximum levels reached in adult lungs. In addition, several organs such as brain, thyroid, and salivary glands express at least 50% of the maximum levels observed in lung. Based on these results it would be predicted that gene delivery using a system targeting the Ly6e receptor would be rather ubiquitous and in particular would target almost as efficiently lung and liver as well as many other organs. This lack of specificity could present potential difficulties in clinical situation, wherein liver targeting should be avoided.

Moreover, it was reported that Syncytin does not generate functional pseudotypes, probably due to improper incorporation into viral particles (Bacquin et al., J. Virol. Doi:10.1128/JVI.00832-17, published only 5 Jul. 2017).

### SUMMARY OF THE INVENTION

Surprisingly, and contrary to what would be expected from the prior art, the authors have found that syncytin may be used to pseudotype LV and as such may be used for targeting gene delivery in lung tissue without risk of liver toxicity. Indeed the murine syncytin-A glycoprotein was used to pseudotype a HIV-1-derived lentiviral vector encoding the luciferase LucII. The pseudotyped LV was injected intravenously to mice and transgene expression to high levels was reproducibly obtained in lung parenchymal cells for long periods of time, at least 3 weeks, with little expression in the liver. Detection of the transgene cassette in the lung of mice, 3 weeks after a single intravenous injection of LV-SynA vector, suggested that stable integrative gene transfer can be achieved, particularly in the lung alveolar cells. Intravenous administration of LV-SynA vector to mice leads to less and very low anti-transgene immune responses compared to LV pseudotyped with VSVg. In addition to murine syncytin A, human syncytin 2 can be used to pseudotype lentiviral vectors to efficiently transduce human lung cells including human primary pulmonary epithelial cells. Cell line expression of mLy6e, reported as the receptor for murine Syncytin A, does not allow to predict the ability to transduce cells by LV pseudotyped with SynA. In addition, there is no correlation between human Ly6e receptor expression mRNA levels and transduction with LV-SynA.

These results provide the proof-of-concept that syncytin can be reliably used for targeted delivery of a therapeutic drug to the lungs including a therapeutic gene or a gene encoding a therapeutic drug. Syncytin, in particular LV pseudotyped with syncytin, can be used to deliver drugs including transgenes in lung alveolar cells following systemic administration. This opens new ways for the treatment of pulmonary diseases such as for example cystic fibrosis, wherein airway blocking mucus prevents access of drugs including gene delivery vectors to lung epithelial cells, in particular alveolar cells.

Thus the present invention relates to a pharmaceutical composition for targeting lung tissue, comprising at least a drug associated to a syncytin protein, for use in the prevention and/or treatment of lung diseases.

### DETAILED DESCRIPTION OF THE INVENTION

Syncytins (also named ERV syncytins) according to the invention refer to highly fusogenic envelope glycoproteins from eutherian mammals, which belong to the family of Endogenous Retroviruses (ERVs). These proteins are encoded by genes, which display a preferential expression in placenta and induce syncytium formation when introduced into cultured cells (Lavialle et al., Phil. Trans. R. Soc. B., 2013, 368:20120507).

Syncytins according to the invention can be selected from human syncytins (e.g.: HERV-W and HERV-FRD), murine syncytins (e.g.: syncytin-A and syncytin-B), syncytin-Ory1, syncytin-Car1, syncytin-Rum1 or their functional orthologs (Dupressoir et al., Proceedings of the National Academy of Sciences of the United States of America, 2005, 102, 725-730; Lavialle et al., Phil. Trans. R. Soc. B., 2013, 368:20120507), and functional fragments thereof comprising at least the receptor binding domain (corresponding to residues 117-144 of Syncytin-1).

By functional orthologs it is intended ortholog proteins encoded by ortholog genes and that exhibit fusogenic properties. Fusogenic properties may be assessed in fusion assays as described in Dupressoir et al. (PNAS 2005). Briefly, cells are transfected for example by using Lipofectamine (Invitrogen) and about 1-2 μg of DNA for 5×10⁵ cells or calcium phosphate precipitation (Invitrogen, 5-20 μg of DNA for 5×10⁵ cells). Plates are generally inspected for cell fusion 24-48 h after transfection. Syncytia can be visualized by using May-Grünwald and Giemsa staining (Sigma) and the fusion index calculated as [(N−S)/T]×100, where N is the number of nuclei in the syncytia, S is the number of syncytia, and T is the total number of nuclei counted.

Human syncytins encompasses HERV-W and HERV-FRD. Functional orthologs of these proteins can be found in Hominidae. HERV-W refers to a highly fusogenic membrane glycoprotein belonging to the family of Human Endogenous Retroviruses (HERVs). HERV-W is an envelope glycoprotein; it is also called Syncytin-1. It has the sequence indicated in Ensembl database, corresponding to Transcript ERVW-1-001, ENST00000493463. The corresponding cDNA has the sequence listed in SEQ ID NO:1. HERV-FRD also refers to a highly fusogenic membrane glycoprotein belonging to the family of Human Endogenous Retroviruses (HERVs). HERV-FRD is an envelope glycoprotein, also called Syncytin-2. It has the sequence indicated in Ensembl database, corresponding to Transcript ERVFRD-1, ENSG00000244476. The corresponding cDNA has the sequence listed in SEQ ID NO:2.

Murine syncytins encompasses murine syncytin-A (i.e.: mus musculus syncytin-A, synA) and murine syncytin-B (i.e.: mus musculus syncytin-B, synB). Functional orthologs of these proteins can be found in the Muridae family. Murine syncytin-A is encoded by the syncytin-A gene. Syncytin-A has the sequence indicated in Ensembl database Syna ENSMUSG00000085957. The corresponding cDNA has the sequence listed in SEQ ID NO:3. Murine syncytin-B is encoded by the syncytin-B gene. Syncytin-B has the sequence indicated in Ensembl databaseSynb ENSMUSG00000047977. The corresponding cDNA has the sequence listed in SEQ ID NO:4.

The syncytin-Ory1 is encoded by the syncytin-Ory1 gene. Functional orthologs of syncytin-Ory1 can be found in the Leporidae family (typically rabbit and hare).

The syncytin-Car1 is encoded by the syncytin-Car1 gene. Functional orthologs of syncytin-Car1 can be found in carnivores mammals from the Laurasiatheria superorder (Cornelis et al., Proceedings of the National Academy of Sciences of the United States of America, 2013, 110, E828-E837; Lavialle et al., Phil. Trans. R. Soc. B., 2013, 368:20120507).

The syncytin-Rum1 is encoded by the syncytin-Rum1 gene. Functional orthologs of syncytin Rum-1 can be found in ruminant mammals.

In the various embodiments of the present invention, the syncytin according to the invention can be typically selected from the group consisting of HERV-W (Syncytin-1) , HERV-FRD (Syncytin-2), syncytin-A, syncytin-B, syncytin-Ory1, syncytin-Car1 and syncytin-Rum1 and their functional orthologs; preferably the syncytin is selected from the group consisting of HERV-W, HERV-FRD, murine syncytin-A, murine syncytin-B and their functional orthologs, more preferably the syncytin is selected from the group consisting of HERV-W, HERV-FRD murine syncytin-A and murine syncytin-B.

In the various embodiments of the present invention, the therapeutic drug is associated to a syncytin protein, directly or indirectly, via covalent or not covalent coupling or bonding using standard coupling methods that are known in the art.

In some embodiments, the drug is covalently coupled to the syncytin protein. For example, the drug can be conjugated to syncytin. Covalent coupling of the drug to syncytin may be achieved by incorporating a reactive group in syncytin protein, and then using the group to link the drug covalently. Alternatively a drug which is a protein can be fused to syncytin to form a fusion protein wherein the syncytin and drug amino acid sequences are linked directly or via a peptide spacer or linker.

In some other embodiments, the drug and syncytin protein are incorporated into a drug delivery vehicle, such as for example a polymer-based or particle-based delivery vehicle including with no limitations micelle, liposome, exosome, dendrimer, microparticle, nanoparticle, virus particle, virus-like particle and others.

As used herein, the term “viral vector” refers to a non-replicating, non-pathogenic virus engineered for the delivery of genetic material into cells. In viral vectors, viral genes essential for replication and virulence have been replaced with heterogenous gene of interest. As used herein, the term “recombinant virus” refers to a virus, in particular a viral vector, produced by recombinant DNA technology.

As used herein, the term “virus particle” or “viral particle” is intended to mean the extracellular form of a non-pathogenic virus, in particular a viral vector, composed of genetic material made from either DNA or RNA surrounded by a protein coat, called the capsid, and in some cases an envelope derived from portions of host cell membranes and including viral glycoproteins.

As used herein, the term “Virus Like Particle” or “VLP” refers to self-assembling, non-replicating, non-pathogenic, genomeless particle, similar in size and conformation to intact infectious virus particle.

In some preferred embodiments, the drug and syncytin protein are incorporated into particles such as for example liposomes, exosomes, microparticles, nanoparticles, virus particles and virus-like particles. The particles are advantageously selected from the group consisting of liposomes, exosomes, virus particles and virus-like particles. Virus particles and virus-like particles include viral capsids and enveloped virus or virus-like particles. Enveloped virus or virus-like particles include pseudotyped virus or virus-like particles. The virus or virus-like particles are preferably from a retrovirus, more preferably a lentivirus. The virus particles are advantageously from a viral vector, preferably a lentiviral vector.

Retrovirus includes in particular gammaretrovirus, spumavirus, and lentivirus. Lentivirus includes in particular human immunodeficiency virus such as HIV type 1 (HIV1) and HIV type 2 (HIV2) and equine infectious anemia virus (EIAV).

Lentivirus-like particles are described for example in Muratori et al., Methods Mol. Biol., 2010, 614, 111-24; Burney et al., Curr. HIV Res., 2006, 4, 475-484; Kaczmarczyk et al., Proc Natl Acad Sci USA., 2011, 108, 16998-17003; Aoki et al., Gene Therapy, 2011, 18, 936-941. Examples of lentivirus-like particles are VLPs generated by co-expressing in producer cells, a syncytin protein with a gag fusion protein (Gag fused with the gene of interest).

The drug and/or syncytin may be, either displayed on the surface of the particles, or enclosed (packaged) into the particles. The syncytin protein is advantageously displayed on the surface of the particles, such as coupled to the particles or incorporated into the envelope of (enveloped) virus particles or virus-like particles to form pseudotyped enveloped virus particles or virus-like particles. The drug is coupled to the particles or packaged into the particles. For example, the drug is coupled to viral capsids or packaged into viral capsids, wherein said viral capsids may further comprise an envelope, preferably pseudotyped with syncytin. In some preferred embodiments, the drug is packaged into the particles pseudotyped with syncytin protein. The drug which is packaged into particles is advantageously a heterologous gene of interest which is packaged into viral vector particles, preferably retroviral vector particles, more preferably lentiviral vector particles.

In some more preferred embodiments, the particles are enveloped virus particles or virus-like particles, preferably enveloped virus particles or virus-like particles pseudotyped with syncytin protein, even more preferably lentivirus vector particles pseudotyped with syncytin protein or lentivirus-like particles pseudotyped with syncytin protein. The enveloped virus particles pseudotyped with syncytin protein, preferably lentivirus vector particles pseudotyped with syncytin protein are advantageously packaging a (heterologous) gene of interest.

In the various embodiments of the present invention the drug is any drug of interest for treating lung diseases by targeted delivery to the cells of the lung tissue, in particular lung parenchymal cells such as lung epithelial cells. Such drugs include with no limitations: anti-infectious drugs such as anti-bacterial, viral, fungal or parasitic drugs; anti-inflammatory drugs, anti-cancer drugs; immunotherapeutic drugs including immunomodulatory, immunosuppressive, anti-histaminic, anti-allergic or immunostimulating drugs; therapeutic proteins including therapeutic antibodies or antibody fragments and genome-editing enzymes, therapeutic peptides, therapeutic RNAs and genes of interest for therapy including therapeutic genes and genes encoding therapeutic proteins, therapeutic peptides, and/or therapeutic RNAs as listed above. In some embodiments, the drug excludes anti-cancer drugs. The drug may be a natural, synthetic or recombinant molecule or agent, such as a nucleic acid, peptide nucleic acid (PNA), protein including antibody and antibody fragment, peptide, lipid including phospholipid, lipoprotein and phospholiprotein, sugar, small molecule, other molecule or agent, or a mixture thereof. Immunosuppressive drugs include for example interleukin 10 (IL10), CTLA4-Ig and other immunosuppressive proteins or peptides. Lipoprotein complex includes in particular pulmonary surfactant. Proteins include surfactant-specific proteins such as protein A (SP-A), SP-B, SP-C and SP-D, in particular SP-B and SP-C. Therapeutic nucleic acids such as therapeutic RNAs include antisense RNAs capable of exon skipping such as modified small nuclear RNAs (snRNAs), guide RNAs or templates for genome editing, and interfering RNAs such as shRNAs and microRNAs.

By “gene of interest for therapy”, “gene of therapeutic interest”, “gene of interest” or “heterologous gene of interest”, it is meant a therapeutic gene or a gene encoding a therapeutic protein, peptide or RNA for treating lung diseases.

The therapeutic gene may be a functional version of a gene or a fragment thereof. The functional version means the wild-type version of said gene, a variant gene belonging to the same family, or a truncated version, which preserves the functionality of the encoded protein. A functional version of a gene is useful for replacement or additive gene therapy to replace a gene, which is deficient or non-functional in a patient. A fragment of a functional version of a gene is useful as recombination template for use in combination with a genome editing enzyme.

Alternatively, the gene of interest may encode a therapeutic protein including a therapeutic antibody or antibody fragment, a genome-editing enzyme or a therapeutic RNA. The gene of interest is a functional gene able to produce the encoded protein, peptide or RNA in cells of the lung tissue, in particular lung parenchymal cells such as lung epithelial cells. The therapeutic protein may be any drug as defined above, such as anti-infectious, anti-inflammatory, anti-cancer and immunotherapeutic drug.

The therapeutic RNA is advantageously complementary to a target DNA or RNA sequence. For example, the therapeutic RNA is an interfering RNA such as a shRNA, a microRNA, a guide RNA (gRNA) for use in combination with a Cas enzyme or similar enzyme for genome editing, or an antisense RNA capable of exon skipping such as a modified small nuclear RNA (snRNA). The interfering RNA or microRNA may be used to regulate the expression of a target gene involved in lung disease. The guide RNA in complex with a Cas enzyme or similar enzyme for genome editing may be used to modify the sequence of a target gene, in particular to correct the sequence of a mutated/deficient gene, or to modify the expression of a target gene involved in lung disease. The antisense RNA capable of exon skipping is used in particular to correct a reading frame and restore expression of a deficient gene having a disrupted reading frame.

The genome-editing enzyme according to the invention is an enzyme or enzyme complex that induces a genetic modification at a target genomic locus. The genome-editing enzyme is advantageously an engineered nuclease which generates a double-strand break (DSB) in the target genomic locus, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease, is usually but not necessarily used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene causing a lung disease.

The gene of interest is advantageously packaged into an enveloped viral vector particle pseudotyped with syncytin protein, preferably a lentivirus vector particle pseudotyped with syncytin protein. The viral vector comprises the gene of interest in a form expressible in lung cells. In particular, the gene of interest is operatively linked to a ubiquitous, tissue-specific or inducible promoter which is functional in lung cells, in particular a strong promoter such as human CMV enhancer combined with human EF1-alpha promoter.

In some preferred embodiments of the present invention, the drug of interest including a gene of interest for treating lung diseases is specific for lung diseases in that it targets a gene or gene product (protein/peptide) involved in lung disease(s) that is specifically expressed in lung cells. In particular, the target gene or gene product is highly expressed in lung cells compared to other cell types. The target genes or gene products include also genes and gene products from lung pathogens such as for example Mycobacterium tuberculosis, Respiratory Syncytial Virus (RSV) and others.

The invention encompasses a pharmaceutical composition comprising two or more drugs associated to a syncytin protein, and/or a composition wherein at least two different syncytin proteins are associated to one or more drugs.

In the various embodiments of the present invention, the pharmaceutical composition, in particular the composition comprising particles as defined previously with syncytin displayed on its surface, and even more preferably lentiviral particles pseudotyped with syncytin packaging a drug of interest including a gene of interest, is used in any targeted therapy of lung diseases by transducing cells of the lung tissue such as in particular lung parenchymal cells such as lung epithelial cells.

The lungs contain the respiratory tract and its lining, which terminate in alveoli, the lung tissue (lung parenchyma) in between, and veins, arteries, nerves and lymphatic vessels.

The lower respiratory tract begins with the trachea and bronchi. These structures are lined with columnar epithelial cells that possess cilia, small frond-like projections. Interspersed with the epithelial cells are epithelial goblet cells which produce mucus, and club cells with actions similar to macrophages. Surrounding these in the trachea and bronchi are cartilage rings, which help to maintain stability. Bronchioles possess the same columnar epithelial lining, but are not surrounded by cartilage rings. Instead, they are encircled by a layer of smooth muscle. The respiratory tract ends in lobules. These consist of a respiratory bronchiole, which branches into alveolar ducts and alveolar sacs, which in turn divide into alveoli.

The epithelial cells throughout the respiratory tract secrete epithelial lining fluid (ELF), the composition of which is tightly regulated and determines how well mucociliary clearance works, which in turn is coated with a layer of surfactant.

Alveoli consist of two types of alveolar cells and alveolar macrophages. The two types of cells are known as type I and type II alveolar cells (also known as pneumocytes). Types I and II make up the walls and alveolar septa. Type I cells provide 95% of the surface area of each alveoli and Type II cells generally cluster in the corners of the alveoli. Type I are squamous epithelial cells that make up the alveolar wall structure. They have extremely thin walls that enable an easy gas exchange. These type I cells also make up the alveolar septa which separate each alveolus. The septa consist of an epithelial lining and associated basement membranes. Type I cells are not able to divide, and consequently rely on differentiation from Type II cells. Type II epithelial cells are larger and they line the alveoli and produce and secrete epithelial lining fluid, and lung surfactant. Type II cells are able to divide and differentiate to Type 1 cells. The composition of the invention allows targeted delivery to the cells of the lung tissue. Typically the composition allows targeted delivery to the lung parenchymal cells such as lung epithelial cells.

In some embodiments of the invention, the pharmaceutical composition of the invention, in particular the composition comprising particles as defined previously with syncytin displayed on its surface, and even more preferably lentiviral vector particles pseudotyped with syncytin packaging a drug or gene of interest, preferably a gene of interest, is used for (targeted) gene therapy of lung diseases.

Gene therapy can be performed by gene transfer, gene editing, exon skipping, RNA-interference, trans-splicing or any other genetic modification of any coding or regulatory sequences in the cell, including those included in the nucleus, mitochondria or as commensal nucleic acid such as with no limitation viral sequences contained in cells.

The two main types of gene therapy are the following:
a therapy aiming to provide a functional replacement gene for a deficient/abnormal gene: this is replacement or additive gene therapy;
a therapy aiming at gene or genome editing: in such a case, the purpose is to provide to a cell the necessary tools to correct the sequence or modify the expression or regulation of a deficient/abnormal gene so that a functional gene is expressed: this is gene editing therapy.

In additive gene therapy, the gene of interest may be a functional version of a gene which is deficient or mutated in a patient, as is the case for example in a genetic disease. In such a case, the gene of interest will restore the expression of a functional gene. More preferably in such embodiment, the composition of the invention preferably comprises a viral vector coding for the gene of interest. Even more preferably, the viral vector is an integrative viral vector such as a retrovirus, notably a lentivirus as previously described.

Gene or genome editing uses one or more gene(s) of interest, such as: (i) a gene encoding a therapeutic RNA as defined above such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); (ii) a gene encoding a genome-editing enzyme as defined above such as an engineered nuclease like a meganuclease, zinc finger nuclease (ZFN), transcription activator-like effector-based nuclease (TALENs), Cas enzyme or similar enzymes; or a combination of such genes, and eventually also a fragment of a functional version of a gene for use as recombination template, as defined above. Gene editing may be performed using non-integrative viral vectors such as non-integrative lentiviral vectors.

Of particular interest are deficient or mutated genes in patients exhibiting a lung disease which, once corrected in cells from the lung tissue, improve the patient's disease or symptoms. The cells from the lung tissue are preferably lung parenchymal cells such as epithelial cells from the respiratory tract or alveolar cells. Examples of mutated genes in genetic disease affecting the lung are the following:
Genes involved in the familial form of chronic obstructive pulmonary disease (COPD) such as (see Seifart C, Plagens A. Genetics of chronic obstructive pulmonary disease. International Journal of Chronic Obstructive Pulmonary Disease. 2007; 2(4):541-550):
   the gene SERPINA1 coding for alpha1-antitrypsin (A1AT), which deficiency is the one proven genetic risk factor for COPD. A1AT is secreted and is a serine protease inhibitor whose targets include elastase, plasmin, thrombin, trypsin, chymotrypsin, and plasminogen activator. A1AT is an acute phase protein that provides the major defense against neutrophil elastase. Defects in this gene can cause emphysema or liver disease. Several transcript variants encoding the same protein have been found for this gene
   the gene SERPINA3 coding for α1-antichymotrypsin (SERPINA3) is capable of inhibiting cathepsin G and mast cell chymase in a reversible fashion. In the SERPINA3 gene two functional SNPs have been associated with low α1-antichymotrypsin levels and COPD;
   the gene coding for the Matrix Metalloproteinases (MMP), such as MMP9, MMP1 and MMP2.
CFTR gene which codes for a chloride transporter found on the surface of the epithelial cells that line the lungs and other organs. CFTR gene defect leads to Cystic fibrosis (CF) one of the most common fatal genetic disease characterized by bronchiectasis. It causes the body to produce thick, sticky mucus that clogs the lungs, leading to infection, and blocks the pancreas, stopping digestive enzymes from reaching the intestines where they are required to digest food. Several hundred mutations have been found in this gene, all of which result in defective transport of chloride, and secondarily sodium, by epithelial cells. As a result, the amount of sodium chloride (salt) is increased in bodily secretions. The severity of the disease symptoms of CF is directly related to the characteristic effects of the particular mutation(s) that have been inherited by the patient.
Defective genes involved in disorders with primary lung pathology, such as the lung Interstitial lung diseases (ILDs) and diffuse parenchymal lung diseases (DLPDs) a heterogeneous collection of over hundred different pulmonary disorders that affect the tissue and spaces surrounding the alveoli. Several mutations are related to disorders with primary lung pathology or with multiple organ pathologies including the lung (see Devine M S, Garcia C K. Genetic Interstitial Lung Disease. Clinics in Chest Medicine. 2012; 33(1):95-110, or Steele M P, Brown K K. Genetic Predisposition to Respiratory Diseases: Infiltrative Lung Diseases. Respiration; international review of thoracic diseases. 2007; 74(6):601-608, as well as Selman M, et al. Surfactant protein A and B genetic variants predispose to idiopathic pulmonary fibrosis. Hum Genet. 2003; 113:542-550 or Nogee L M, et al. Allelic heterogeneity in hereditary surfactant protein B (SP-B) deficiency. Am J Respir Crit Care Med. 2000; 161:973-981), such as:

Gene Associates with Surfactant Dysfunctions:
SFTPB gene, which mutations in have been associated with absent SP-B and found in patients with Surfactant Metabolism Dysfunction 1, a severe respiratory distress in neonates and infants;
SFTPC gene, which mutations have been associated with Lack of SP-C and ER stress and found in patients with Surfactant Metabolism Dysfunction 2, a severe respiratory distress in neonates and infants;
SFTPA2 gene, which mutations have been associated with ER stress of alveolar epithelium and found in patients with Familial Pulmonary Fibrosis;
SFTPD gene, which mutations have been associated with COPD and atopy in asthma (Fakih et al., Respirology, 2017 Sep. 28.doi/10.1111/resp.13193);
ABCA3 gene, which mutations have been associated with Defective transport of phospholipid into lamellar bodies and found in patients with Surfactant Metabolism Dysfunction 3, a severe respiratory distress in neonates and infants;
CSF2RA gene (which mutations have been associated with Defective GM-CSF signaling), CSF2RB have been found in patients with Surfactant Metabolism Dysfunction 4, a severe respiratory distress in neonates and infants;
SLC34A2 gene, which mutations have been associated with Reduced phosphate clearance from alveolar space and found in patients with pulmonary Alveolar Microlithiasis. Although this protein can be found in several organs and tissues in the body, it is located mainly in the millions of small air sacs (alveoli) in the lungs, specifically in alveolar type II cells which produce surfactant.

Other Genes of Interest:
TERT gene, which mutations have been associated with Telomere shortening and found in patients with Familial Pulmonary Fibrosis;
TERC gene, which mutations has been found in patients with Familial Pulmonary Fibrosis;
DKC1, TERC, TERT, TINF2 genes, which mutations have been associated with telomere shortening and related to dyskeratosis congenita;
NF1 gene, which mutations have been associated with loss of function of tumor suppressor in patients with neurofibromatosis of type I;
TSC1 gene (which mutations have been associated with proliferation of LAM cells) and TSC2 which mutations have been found in patients with Tuberous Sclerosis/LAM;
FLCN gene, which mutations have been associated with Loss of folliculin have been found in patients with Birt-Hogg-Dube Syndrome;
HPS1 gene, which mutations have been associated with defect in cytoplasmic organelles) and HSP4 gene which mutations have been found in patient with Hermansky-Pudlak syndrome;
GBA gene, which mutations have been associated with Deficiency of acid β-glucosidase have been found in patients with Gaucher disease, type I;
SMPD1 gene, which mutations have been associated with Deficiency of acid sphingomyelinase, and found in patients with Niemann-Pick disease, type B;
SLC7A7 gene, which mutation have been associated with Defect of cationic amino acid transport and found in patients with Lysinuric protein intolerance;
Defective genes involved in primary pulmonary hypertension, causing high pulmonary arterial pressure, vascular remodeling and premature death, such as:
   SMAD9 gene, coding for a member of the SMAD family, which transduces signals from TGF-beta family members. The encoded protein is activated by bone morphogenetic proteins and interacts with SMAD4. A heterozygous truncating mutation in the SMAD9 gene (R294X) has been identified in patient with primary pulmonary hypertension-2 (PPH2).
   KCNK3 gene encoding a pH-sensitive potassium channel in the 4-transmembrane/2-pore domain superfamily. Several substitutions at highly conserved residues, including E182K, V221L, G97R and G203D, were identified in patients with primary pulmonary hypertension-4 (PPH4).

CAV1 gene which mutations have been associated with primary pulmonary hypertension-3 (PPH3).

Such genes may be targeted in the lung tissue in replacement gene therapy, wherein the gene of interest is a functional version of the deficient or mutated gene.

Alternatively, these genes could be used as target for gene editing. A specific example of gene editing would be the treatment of cystic fibrosis: in such a disease, point deletion (F508Del) or point mutations such as G551D, G542X (L265P) of the CFTR gene has been observed in some patient populations. Thus, by gene editing a correct version of this gene in afflicted patients, this may contribute to effective therapies against this disease. Other genetic diseases of the lung as listed above could be treated by gene editing using the same principle.

Examples of mutated genes in genetic diseases with primary lung pathology or with multiple organ pathologies including severe lung pathology in particular those expressed in lung epithelial cells which lead to the production of non-functional epithelial lining fluid or lung surfactant which could be treated by expressing or correcting the causal gene in epithelial cells, are the following: SERPINA3, SERPINA1, MMP (notably MMP1, MMP2 and MMP9), CFTR, SFTPB, SFTPC, ABCA3, CSF2RA, TERT, TERC, SFTPA2, SLC34A2, DKC1, TERC, TERT, TINF2, NF1, TSC1, FLCN, STAT3, HPS1, GBA, SMPD1, SLC7A7, SMAD9, KCNK3 and CAV1.

More preferably a gene of interest can be selected from the group comprising SERPINA3, SERPINA1, MMP, CFTR, SFTPB, SFTPC, ABCA3, CSF2RA, TERT, TERC, SFTPA2, SLC34A2, SMAD9, KCNK3 and CAV1 which are specifically associated with diseases affecting the lung including cystic fibrosis, chronic obstructive pulmonary disease (COPD), various Interstitial lung diseases (ILDs) or diffuse parenchymal lung diseases and primary pulmonary hypertension.

Gene selected from the group comprising DKC1, TERC, TERT, TINF2, NF1, TSC1, FLCN, STAT3, HPS1, GBA, SMPD1 and SLC7A7 are associated with genetic disorders affecting multiple organs, including the lung.

In one embodiment, a gene of interest can also be selected from genes which are specifically expressed in the lung, such as more particularly the group comprising SERPINA3, SERPINA1, CFTR, SFTPB, SFTPC, SFTPA2, and ABCA3.

Gene therapy could also be used for treating asthma, by correcting surfactant protein D gene mutations causing COPD and atopy in asthma (Fakih et al., Respirology 2017) or by expressing molecules, such as antibodies or antibody fragments, blocking IL4, IL-13 or IL-23 in lungs or molecules regulating eosinophils, mast cells, or Th2 inflammation in lung.

Replacement or additive gene therapy could also be used to treat cancer, notably lung cancer. Genes of interest in cancer could regulate the cell cycle or the metabolism and migration of the tumor cells, or induce tumor cell death. For instance, inducible caspase-9 could be expressed in epithelial cells of the lung tissue to trigger cell death, preferably in combination therapy to elicit durable anti-tumor immune responses.

In gene therapy, it might be possible to use the composition of the invention as previously described and more particularly, the stable lentiviral particles pseudotyped with syncytin as per the invention in therapy for lung tissue engineering, preferably endogenous pulmonary stem cells engineering, by transducing said cells (Nichols J E, Niles J A, Cortiella J. Design and development of tissue engineered lung: Progress and challenges. Organogenesis. 2009, 5, 57-61).

It could also be possible to insert sequences favoring gene splicing, expression or regulation or gene editing. Tools such as CRISPR/Cas9 may be used for this purpose. This could be used to modify gene expression in lung parenchymal cells such as lung epithelial cells, in the case of auto-immunity or cancer, or to perturb the cycle of viruses in such cells. In such cases, preferably, the heterologous gene of interest is chosen from those encoding guide RNA (gRNA), site-specific endonucleases (TALEN, meganucleases, zinc finger nucleases, Cas nuclease), DNA templates and RNAi components, such as shRNA and microRNA.

To treat infectious diseases of the lung, the gene of interest may also target essential components of the lung pathogen life cycle. For example, the gene of interest can encode an antibody or antibody-like molecule capable of recognizing specifically a lung pathogen and blocking its life cycle or its effects.

By “infectious diseases”, it is meant diseases which are caused by pathogenic microorganisms, such as bacteria, viruses, parasites or fungi; the diseases can be spread, directly or indirectly, from one person to another.

The pharmaceutical composition comprising stable pseudotyped lentiviral particles according to the invention could be used together or sequentially to target the same cells. This could be an advantage in strategies such as gene editing, in which multiple components of the gene editing platform need to be added to the cells.

In some other embodiments of the invention, the pharmaceutical composition of the invention comprising a drug associated to a syncytin protein, in particular the composition comprising particles as defined previously with syncytin displayed on its surface, and even more preferably lentiviral particles pseudotyped with syncytin packaging a drug or gene of interest, preferably a gene of interest, is used for immunomodulation or to modulate lung transplant tolerance. In particular, the composition is administered to a lung transplant donor for the prevention of lung transplant rejection. For these uses, the drug is in particular an immunosuppressive drug such as IL-10, CTLA4-Ig or other immunosuppressive peptides, or VEGF mutants that improve lymphangiogenesis (Cui et al. J. Clin. Invest. 2015, Nov. 2; 125(11):4255-68.) and the gene of interest is a gene encoding said immunosuppressive drugs or VEGF mutants.

In some other embodiments of the invention, the pharmaceutical composition of the invention comprising a drug associated to a syncytin protein, in particular the composition comprising particles as defined previously with syncytin displayed on its surface, and even more preferably lentiviral particles pseudotyped with syncytin packaging a drug or gene of interest, preferably a gene of interest is used for treating fibrosis or amyloidosis with competing proteins.

In some other embodiments, the pharmaceutical composition of the invention comprising pulmonary surfactant or surfactant complexes delivered by syncytin-containing virus-like particles are administered to newborns or premature babies in surfactant replacement therapy for the prevention of lung problems or diseases (see for example Poulain F R, Clements J A. Pulmonary surfactant therapy. Western Journal of Medicine. 1995; 162, 43-50). In particular, the pharmaceutical composition comprises surfactant protein(s) or RNA(s) encoding surfactant protein(s) packaged into enveloped lentivirus-like particles displaying syncytin on their surface.

In the various embodiments of the present invention, the pharmaceutical composition comprises a therapeutically effective amount of drug associated to syncytin protein.

In the context of the invention, the term “treating” or “treatment”, as used herein, means reversing, alleviating or inhibiting the progress of the disorder or condition to which such term applies, or reversing, alleviating or inhibiting the progress of one or more symptoms of the disorder or condition to which such term applies.

Likewise, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present invention, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with enveloped viruses and do not prevent virus entry into target cells. In all cases, the form must be sterile and must be fluid to the extent that easy syringability exists. It must be stable under the conditions of manufacture and storage and must be preserved against the contaminating action of microorganisms, such as bacteria and fungi. An example of an appropriate solution is a buffer, such as phosphate buffered saline (PBS).

The invention provides also a method for treating a lung disease, comprising: administering to a patient a therapeutically effective amount of the pharmaceutical composition as described above. The lower immunogenicity of LV pseudotyped with syncytin is expected to allow long-term gene expression in cells from lung tissue by repeated administration of the pharmaceutical composition.

As used herein, the term “patient” or “individual” denotes a mammal. Preferably, a patient or individual according to the invention is a human.

The pharmaceutical composition of the present invention, in particular, the composition comprising particles as defined previously with syncytin displayed on its surface, and even more preferably lentiviral particles pseudotyped with syncytin packaging a drug of interest including a gene of interest, is generally administered according to known procedures, at dosages and for periods of time effective to induce a therapeutic effect in the patient.

The administration may be by injection, oral or local (respiratory) administration. The injection may be subcutaneous (SC), intramuscular (IM), intravenous (IV), intraperitoneal (IP), intradermal (ID) or else. Preferably, the administration is by injection, inhalation or broncho-alveolar lavage. Preferably the injection is intravenous. The inhalation is advantageously done by nebulisation.

In the various embodiments of the present invention, the lung diseases may be advantageously selected from the group consisting of: genetic diseases affecting the lung such as typically cystic fibrosis and alpha-1 antitrypsin deficiency; infectious diseases affecting the lung such as tuberculosis, Respiratory Syncytial Virus (RSV) infection or other pneumonia (such as interstitial pneumonia); somatic and inflammatory diseases of the lung such as cancer (more particularly lung cancer and lung metastasis), asthma, chronic obstructive pulmonary disease (COPD), pulmonary fibrosis, edema, emphysema or hypertension, acute respiratory distress syndrome (ARDS), pneumoconiosis such as from asbestos exposure, auto-immune diseases of the lung such as nonspecific interstitial pneumonitis, any other interstitial lung diseases (ILDs), or diffuse parenchymal lung diseases (DLPDs), prevention of lung transplant rejection and prevention of lung problems or diseases in newborns and premature babies. In some embodiments, the lung diseases exclude cancer. In some embodiments, Syncytin A, preferably lentiviral vector particles pseudotyped with syncytin A packaging a drug or gene of interest, preferably a gene of interest, are used for treating lung cancers. In some preferred embodiment, the lung diseases are selected from the group consisting of: genetic diseases affecting the lungs; infectious diseases affecting the lungs; inflammatory or auto-immune diseases of the lungs, asthma, chronic obstructive pulmonary disease, pulmonary fibrosis, oedema, emphysema or hypertension, acute respiratory distress syndrome, pneumoconiosis, interstitial lung diseases or diffuse parenchymal lung diseases, prevention of lung transplant rejection and prevention of lung problems or diseases in newborns and premature babies.

The present invention relates also to the use of a composition of the invention comprising a drug associated to a syncytin protein, in particular the composition comprising particles as defined previously with syncytin displayed on its surface, and even more preferably lentiviral particles pseudotyped with syncytin packaging a drug or gene of interest, preferably a gene of interest, for non-therapeutic purposes. Non-therapeutic purposes include increasing the lung capacity of a healthy subject, in particular to breathe underwater.

The present invention relates also to the use of a composition of the invention for the preparation of lung transplant or post-mortem conservation of lung tissue. In such embodiment the drug may be for example selected from antioxydants.

The invention also relates to an in vivo or ex vivo method of imaging lung tissue, comprising the administration of a syncytin protein associated to an imagining agent to an individual. Advantageously, said syncytin protein associated to an imaging agent is a lentiviral particle pseudotyped with syncytin, which incorporates an imaging agent. More preferably said imaging agent is coupled to the viral capsid or enclosed into said viral capsid.

The invention relates also to a pharmaceutical composition for targeting lung disease, as defined above, comprising a drug of interest specific for lung disease associated to syncytin protein, wherein the drug of interest including gene of interest, targets a gene or gene product (protein/peptide) involved in lung disease(s) that is specifically expressed in lung cells, as defined above.

In some preferred embodiments, the pharmaceutical composition comprises a gene of interest for gene therapy of lung diseases. Preferably, the gene of interest targets a gene responsible for a genetic disease affecting the lungs, such as in particular selected from the group comprising cystic fibrosis, chronic obstructive pulmonary diseases (COPD), Interstitial lung diseases (ILDs), diffuse parenchymal lung diseases and primary pulmonary hypertensions.

The target gene responsible for a genetic disease can be selected from the group comprising SERPINA3, SERPINA1, MMP, CFTR, SFTPB, SFTPC, ABCA3, CSF2RA, TERT, TERC, SFTPA2, SLC34A2, SMAD9, KCNK3 and CAV1 which are specifically associated with diseases affecting the lung notably selected from the group comprising SERPINA3, SERPINA1, CFTR, SFTPB, SFTPC, SFTPA2, and ABCA3 which are specifically expressed in the lung. The gene of interest is suitable for gene therapy of said genetic disease by gene replacement or gene editing, as defined above.

In some other preferred embodiments, the pharmaceutical composition comprises a gene of interest targeting an essential gene of a lung pathogen. The pathogen can be selected from the group comprising Mycobacterium tuberculosis, Respiratory Syncytial Virus (RSV) and others.

In the various embodiments, the pharmaceutical composition preferably comprises particles with syncytin displayed on their surface, and even more preferably lentiviral particles pseudotyped with syncytin packaging a gene of interest for gene therapy of lung diseases by targeting specifically a gene expressed in the lungs.

The practice of the present invention will employ, unless otherwise indicated, conventional techniques, which are within the skill of the art. Such techniques are explained fully in the literature.

In the various embodiments, viral particles, in particular viral vector particles, and virus-like particles may be produced using standard recombinant DNA technology techniques.

In particular, stable pseudotyped lentiviral particles including a heterologous gene of interest for use in the invention may be obtained by a method comprising the following steps:
a) transfecting at least one plasmid in appropriate cell lines, wherein said at least one plasmid comprises the heterologous gene of interest, the retroviral rev, gag and pol genes, and a nucleic acid coding for an ERV syncytin;
b) incubating the transfected cells obtained in a), so that they produce the stable lentiviral particles pseudotyped with an ERV syncytin, respectively, and packaging the heterologous gene of interest; and
c) harvesting and concentrating the stable lentiviral particles obtained in b).

The method allows obtaining high physical titers, as well as high infectious titers, of stable pseudotyped lentiviral particles including a heterologous gene of interest. Preferably, step c) of the method comprises harvesting, concentrating and/or purifying the stable lentiviral particles produced in step b), from the supernatant. Thus, preferably, the concentration of step c) comprises centrifugating and/or purifying the harvested stable lentiviral particles obtained in b). Said harvest may be performed according to well-known methods in the art. Preferably, the lentiviral vectors are harvested before fusion of the transfected cells, more preferably between 20 hours and 72 hours post-transfection, preferably after 24 hours. Preferably, the harvesting step consists of a single lentivirus harvest, preferably implemented between 20 and 72 hours post-transfection, preferably between 20 and 30 hours post-transfection, more preferably after 24 hours.

In step a), appropriate cell lines are transfected with at least one plasmid. Preferably, the transfection is a transient transfection. Preferably, appropriate cell lines are transfected with at least one, two, three or four plasmids. These cell types include any eukaryotic cell which support the lentivirus life cycle. Preferably, the appropriate cell lines are stable cell lines or cell lines refractory to the catastrophic consequences of the fusogenic effects of syncytins, so as to continue growing while producing the particles. Said appropriate cell lines are mammalian cell lines, preferably human cell lines. Representative examples of such cells include Human Embryonic Kidney (HEK) 293 cells and derivatives thereof, HEK293 T cells, as well as subsets of cells selected for their ability to grow as adherent cells, or adapted to grow in suspension under serum-free conditions. Such cells are highly transfectable.

The appropriate cell lines may already be expressing at least one, and at most four of the five sequences which are the heterologous gene of interest, the retroviral rev, gag and pol genes, and the nucleic acid coding for an ERV syncytin such as HERV-W, HERV-FRD or murine syncytinA, preferably in inducible form. In such a case, step a) comprises transfecting said cell line with at least one plasmid comprising at least one sequence which is not already expressed in said cell line. The plasmid mixture, or the single plasmid (if only one plasmid is used) is chosen such that, when transfected into said cell lines in step a), said cell lines express all five above sequences. For example, if the appropriate cell line expresses the retroviral rev, gag and pol genes, then the plasmid or mixture of plasmids to be transfected comprises the remaining sequences to be expressed, i.e. the heterologous gene of interest and the nucleic acid coding for an ERV syncytin such as HERV-W, HERV-FRD or murine syncytinA.

When one single plasmid is used, it comprises all the 5 sequences of interest, i.e.:
the heterologous gene of interest,
the rev, gag and pol genes, and
a nucleic acid coding for an ERV syncytin as previously described and notably coding for HERV-W, HERV-FRD or the murine syncytinA.

When two or three plasmids are used (plasmid mixture), each of them comprises some of the sequences of interest listed in the previous paragraph, so that the plasmid mixture comprises all the above cited sequences of interest.

Preferably four plasmids are used, and the quadritransfection comprises the following:
the first plasmid comprises the gene of interest,
the second plasmid comprises the rev gene,
the third plasmid comprises the gag and pol genes, and
the fourth plasmid comprises a nucleic acid coding for an ERV syncytin as previously described and notably coding for HERV-W, HERV-FRD or the murine syncytin-A.

Said quadritransfection is preferably performed with specific ratios between the four plasmids. The molar ratio between the different plasmids can be adapted for optimizing the scale-up of the production. The person skilled in the art is able to adapt this parameter to the specific plasmids he uses for producing the lentivirus of interest. In particular, the weight ratios of the first, second, third, fourth plasmids are preferably (0.8-1.2):(0.1-0.4); (0.5-0.8):(0.8-1.2), more preferably around 1:0.25; 0.65; 0.9.

The rev, gag and pol genes are retroviral, preferably lentiviral. Preferably, they are HIV genes, preferably HIV-1 genes, but could be also EIAV (Equine Infectious Anemia Virus), SIV (Simian immunodeficiency Virus), Foamy Virus, or MLV (Murine Leukemia Virus) virus genes.

The nucleic acid coding for the ERV syncytin, such as an ERV syncytin as previously defined and more preferentially coding for HERV-W, HERV-FRD or the murine syncytin-A is a DNA or cDNA sequence. Preferably, it corresponds to the cDNA sequence respectively listed in SEQ ID NO:1, 2 or 3, or to a sequence presenting at least 80%, preferably at least 90%, more preferably at least 95%, more preferably at least 99% identity with such SEQ ID NO:1, 2, or 3 respectively. Preferably, step a) comprises the transfection of at least the plasmid comprising, preferably consisting of, the cDNA sequence listed in SEQ ID NO:5 or 6.

The term “identity” refers to the sequence similarity between two polypeptide molecules or between two nucleic acid molecule. When a position in both compared sequences is occupied by the same base or same amino acid residue, then the respective molecules are identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wis.) pileup program, or any of sequence comparison algorithms such as BLAST, FASTA or CLUSTALW.

The plasmids encoding the envelope glycoproteins which may be used are known to those skilled in the art such as the commercially available pCDNA3, backbone or any other plasmid cassette using a similar expression system, for instance using the CMV promoter such as the pKG plasmid described in Merten et al. (Human gene therapy, 2011, 22, 343-356).

According to step a), various techniques known in the art may be employed for introducing nucleic acid molecules into cells. Such techniques include chemical-facilitated transfection using compounds such as calcium phosphate, cationic lipids, cationic polymers, liposome-mediated transfection, such as cationic liposome like Lipofectamine (Lipofectamine 2000 or 3000), polyethyleneimine (PEI), non-chemical methods such as electroporation, particle bombardment or microinjection. The transfection of step a) is preferably carried out using calcium phosphate.

Typically, step a) may be performed by transient transfection of 293T cells with 4 plasmids (quadritransfection), in the presence of calcium phosphate. The 4 plasmids are preferably: a pKL plasmid expressing the HIV-1 gag and pol genes, a pK plasmid expressing HIV-1 rev gene, a pCCL plasmid expressing the heterologous gene of interest under control of a cellular promoter such as the human phosphoglycerate kinase (PGK) promoter and a pCDNA3 plasmid expressing an ERV syncytin, such as an ERV syncytin as previously defined and more preferentially expressing HERV-W (Syncytin-1), HERV-FRD (Syncytin-2) or the murine syncytin-A (Syncytin-A) glycoproteins from a CMV promoter.

Then, after step a), the method comprises a step b) of incubating the transfected cells obtained in a), so that they produce, preferably in the supernatant, the lentiviral particles pseudotyped with an ERV syncytin, such as an ERV syncytin as previously defined and more preferentially pseudotyped with HERV-W, HERV-FRD or the murine syncytin-A including the heterologous gene of interest. Indeed, once step a) is performed, incubation of the obtained cells is performed. This leads to the production in the supernatant of the stable lentiviral particles, which are pseudotyped with an ERV syncytin, such as an ERV syncytin as previously defined and more preferentially pseudotyped with HERV-W, HERV-FRD or the murine syncytin-A and which include the heterologous gene of interest.

After transfection, the transfected cells are thus allowed to grow for a time which may be comprised between 20 and 72 hours post-transfection, in particular after 24 hours.

The medium used for culturing the cells may be a classical medium, such as DMEM, comprising a sugar, such as glucose. Preferably, the medium is a serum-free medium. Culture may be carried out in a number of culture devices such as multistack systems or bioreactors adapted to the culture of cells in suspension. The bioreactor may be a single-use (disposable) or reusable bioreactor. The bioreactor may for example be selected from culture vessels or bags and tank reactors. Non-limiting representative bioreactors include a glass bioreactor (e.g. B-DCU® 2L-10L, Sartorius), a single-use bioreactor utilizing rocking motion agitation such as wave bioreactor (e.g. Cultibag RM® 10L-25L, Sartorius), single use stirrer tank bioreactor (Cultibag STR® 50L, Sartorius), or stainless steel tank bioreactor.

After incubation, the obtained stable lentiviral particles are harvested and concentrated; this is step c). Preferably, the stable lentiviral particles obtained in b) are harvested before fusion of the transfected cells, more preferably 24h post-transfection. Preferably, the stable lentiviral particles present in the supernatant obtained in b) are centrifugated and/or purified. Said concentration step c) may be performed by any known method in the art, such as by centrifugation, ultrafiltration/diafiltration and/or chromatography.

The supernatant may be centrifugated at a speed comprised between 40000 and 60000 g, during 1 h to 3 h, at a temperature comprised between 1° C. and 5° C., so as to obtain a centrifugate of stable pseudotyped viral particles. Preferably, the centrifugation is performed at a speed of 45000 to 55000 g, during 1 h 30 to 2 h 30, at a temperature of 2° C. to 5° C., preferably around 4° C. At the end of this step, the particles are concentrated in the form of a centrifugate, which may be used.

Step c) may be chromatography, such as an anion exchange chromatography, or an affinity chromatography. The anion exchange chromatography may be preceded or followed by a step of ultrafiltration, in particular an ultrafiltration/diafiltration, including tangential flow filtration. The anion exchange chromatography is for example a weak anion exchange chromatography (including DEAE (D)—diethylaminoethyl, PI—polyethylenimine).

The invention will now be exemplified with the following examples, which are not limitative, with reference to the attached drawings in which:

### FIGURE LEGENDS

FIG. 1: Bioluminescence analysis following SynA-LV Luc2 delivery.

Bioluminescence of mice injected with LucII-expressing vectors comparing vectors pseudotyped with Syncytin A (SyA) or VSVg versus control PBS. Two representative mice per group. Mice 1 & 2 were injected with PBS, mice 10 & 11 were injected with LV-SyA-LucII and mice 12 & 15 were injected with LV-VSVg-LucII. Bioluminescence analyses were performed at day 16 post IV injection with the IVIS Lumina apparatus. The whole body luminescence is measured in photon/sec. Bioluminescence is measured on the front and on the back of the mice using a contour mask of the animal.
PBS group: The signal of mouse 1 (S1) from the back is 3.531.10⁵photons/second, and from the front 1.446.10⁴ photons/second. For mouse 2 (S2), the signal from the back is 3.351.10⁵photons/second and from the front 1.163.10⁴ photons/second.
LV-SyA-LucII group: For the mouse 10 (S10), the signal from the back is 3.952.10⁷photons/second and from the front 1.019.10⁷ photons/second. For mouse 11 (S11) the signal from the back is 4.344.10⁷ photons/second and from the front 3,573.10⁷ photons/second.
LV-VSVg-LucII group: For mouse 12 (S12), the signal from the back is 3.052.10⁸ photons/second and from the front 6.636.10⁸photons/second. For mouse 15 (S15), the signal from the front is 5.478.10⁸ photons/second and from the front 8,311.10⁸ photons/second.

FIG. 2: Quantification of the bioluminescence signal in the lungs following Syncytin A-LV LucII systemic delivery.

LV-Syncytin A or -VSVg encoding LucII transgene (LV-SynA (n=20) or LV-VSVg (n=4)) was injected intravenously into albinos C57Bl/6 mice at a dose of 5×10⁵ ig (infectious genome)/mouse, PBS is injected as a control (n=11). Bioluminescence signal is measured in the lungs (photons/sec) in individual mice over time, at day 7, 14 and 21 post-injection (upon exposure of the back). Each dot represents a mouse.

FIG. 3. Biodistribution of gene transfer following syncytinA-mediated gene delivery.

LV-Syncytin A encoding LucII transgene (LV-SA-LucII; n=6) were injected intravenously into albinos C57Bl/6 mice (3×10⁵ ng p24/mouse). As control a LV-VSVg encoding the same transgene was used (LV-VSVg-LucII; n=4), as well as PBS (n=3). The Vector Copy Number per diploid cell (VCN) was measured by qPCR in the different organs recovered at the time of sacrifice of the mice at week 3. The grey zone indicated the limit of quantification of the technique.

FIG. 4: PCR around the PSI sequence of the integrated provirus in lungs of mice injected with a single dose of LV-SynA vector

LV-Syncytin A or -VSVg encoding LucII transgene (LV-SynA (n=6) or LV-VSVg (n=4)) was injected intravenously into albinos C57Bl/6 mice at a dose of 5×10⁵ ig (infectious genome)/mouse, PBS is injected as a control (n=2). Genomic DNA is extracted from total lung cells at 3 weeks post-injection. A PCR is performed on these gDNA, amplifying the PSI sequence of the integrated provirus, the expected fragment of PSI is at 489 bp.

FIG. 5: Representative immunohistostaining of luciferase in the lung following syncytinA-mediated gene delivery.

A-LV-Syncytin A encoding LucII transgene (LV-SA-LucII; n=6) were injected intravenously into albinos C57Bl/6 mice (3×10⁵ ng p24/mouse). As control PBS (n=3) was used. Sections of frozen lung (representative of 3 mice tested in such way) were stained with an anti-luciferase antibody and revealed with a secondary antibody coupled with AlexaFluor 594. Nuclei were counterstained by DAPI. Sections were visualized with the confocal microscope Leica SP8. The top panels of FIG. 5A correspond to a lung section from a PBS-treated control animal and the bottom panel corresponds to a lung section from a LV-SYnA-Luc2-treated mouse. The left panels of FIG. 5A show the luciferase immunostaining and the right panel shows the same section with the DAPI stain which shows all cell nuclei on the section.

B-C LV-Syncytin A encoding LucII transgene (LV-SA-LucII) was injected intravenously into albinos C57Bl/6 mice (5×105 IG/mouse). Three weeks after injection, mice were sacrificed, lungs were fixed, paraffin-embedded and sectioned for immunostaining. Images in (B) and (C) are representative of 8 mice tested in such way. Lung sections were stained with DAPI (B) to detect all cell nuclei defining the presence of alveoli; and with an anti-luciferase antibody revealed with a secondary antibody coupled with AlexaFluor 594 to detect the luciferase-expressing cells and in particular the epithelial cells lining the lung alveoli (C). Controls included mice injected with PBS in the same conditions (n=11 mice). Immunostaining of lungs in PBS-injected mice showed DAPI+cells without detectable luciferase (data not shown).

FIG. 6: Reduced immune response against transgene following systemic delivery using LV-SynA, compared to LV-VSVg, as measured using Elispot anti-IFNg and CBA.

Six-week-old C57BL/6 mice were injected intravenously (IV) into the tail vein with PBS, 7.5.10⁵ ig (infectious genome)/mouse of LV-SynA_GFP-HY or LV-VSVg_GFP-HY vectors.

(A) Twenty-one days later, spleen cells were harvested to measure Dby-specific CD4+T cell and Uty-specific CD8+ T cell response by γIFN-ELISPOT following peptide in vitro stimulation. Data represent one experiment including 3 mice per group.

(B) For the titration of cytokines secreted by T cells. Three weeks after the immunization, total splenocytes were re-stimulated in vitro by Dby, Uty peptides, or Concanavalin A (conA) as positive control. After 36 h of culture, supernatants were removed and titrated for the indicated cytokines (3 mice/group/experiment). Each point represent an individual measurement with at least 2 measurement per mice.

FIG. 7: Human lung MRC5 and WI26VA4 cells transduction with LV-SynA (n=2 experiments).

Vector copy number per cell were determined by qPCR following transduction of two cell lines with increasing concentrations (10⁵, 5×10⁵ infectious genome (ig)/mL) of a LV pseudotyped with Syncytin-A. As a positive control, cells were transduced with 10⁶ infectious units/mL of a LV pseudotyped with VSVg. The negative control consisted of non-transduced cells. Results of two experiments.

FIG. 8: Human lung MRC5 cells transduction with LV-Syncytins (-A, -B, -1 or -2) 7 days post-transduction (n=3 experiments).

Vector copy number per cell was determined at 7 days post-transduction by qPCR following transduction of MRC5 cells with a concentration of 10⁵ IG/mL of LV pseudotyped with Syncytins (-1, -2, -A or -B), in presence of Vectofusin-1® (12μg/mL). As a positive control, cells were transduced with 10⁶ IG/mL of a LV pseudotyped with VSVg. The negative control consisted of non-transduced cells. Results of two experiments for LV-SynB, LV-Syn1 and LV-Syn2, or three experiments for LV-SynA and LV-VSVg.

FIG. 9: Human Small Airway Epithelial Cells transduction with LV-Syncytins (-A, -1 or -2) 7 days post-transduction (n=5 experiments).

Vector copy number per cell was determined at 7 days post-transduction by qPCR following transduction of Human Small Airway Epithelial Cells (Primary Small Airway Epithelial Cells; Normal, Human (ATCC® PCS301010™) with a concentration of 10⁵ ig/mL of LV pseudotyped with Syncytins (-A, -1 or -2) in presence of Vectofusin-1® (12 μg/mL). As a positive control, cells were transduced with 10⁶ IG/mL of a LV pseudotyped with VSVg and confirmed the ability to transduce these cells (0.75 vector copy per cell was found) and to detect the transgene in these cells (data not shown). The negative control consisted of un-transduced cells (no vector). Results are averages of five experiments for LV-SynA, and three experiments for LV-Syn1, LV-Syn2 (and LV-VSVg).

FIG. 10: Comparison between the level of expression of mLy6e mRNA and the level of transduction on different cell lines.

(A) mRNA were extracted from different cell lines (A20IIA, C2C12, NIH/3T3) and converted into cDNA to perform a qRT-PCR on mLy6e, using PO as a housekeeping gene. Relative levels were calculated with the formula abundance=2^{-ΔCt}. The qRT-PCR was validated by testing total cells from the lung, spleen or bone marrow of C57BL/6 mice which confirmed that the mLy6e expression level was the highest in lung cells, as published by Bacquin et al 2017 (data not shown).

(B) The same cell lines as in FIG. 10 (A) were transduced with LV-Syncytin A vectors encoding ΔNGFR at a dose of 10⁵ IG/mL. The level of transduction was analysed by flow cytometry at 7 days post-transduction.

FIG. 11: Comparison between the level of expression of hLy6e mRNA and the level of transduction on different cell lines.

(A) mRNA were extracted from different cell lines (HEK293T, HCT116, HT1080, WI26VA4, Jurkat, RAJI and MRC5) and converted into cDNA to perform a qRT-PCR on hLy6e, using PO as a housekeeping gene. Relative levels were calculated with the formula abundance=2^{-ΔCt}. The numbers indicated on the bar graph are the actual values of mRNA expression.

(B) The same cell lines as in FIG. 10 (A) were transduced with LV-Syncytin A vectors encoding ΔNGFR at a dose of 10⁵ IG/mL. The level of transduction was analysed by flow cytometry at 7 days post-transduction.

### EXAMPLE 1

### Production of Stable and Infectious LV-SynA Particles

Materials and Methods

Cell Lines

Human embryonic kidney 293T cells were cultured at 37° C., 5% CO2 in Dulbecco's modified Eagle's medium (DMEM+glutamax) (Life Technologies, St-Aubin, France) supplemented with 10% of heat inactivated fetal calf serum (FCS) (Life Technologies).

Cloning of Syncytin A and Production of LV-Syn A.

a. Generation of a Plasmid Expressing Murine Syncytin-A.

Murine syncytin-A cDNA was cloned into a pCDNA3 plasmid using standard techniques.

b. Production of Syn-A-Pseudotyped Lentiviral Vectors.

HEK293T cells were co-transfected with the following 4 plasmids (quantities per flask), using calcium phosphate: pKLgagpol expressing the HIV-1 gagpol gene (14.6 μg), pKRev expressing HIV-1 rev sequences (5.6 μg), pcDNA3.1SynA (20 μg), and gene transfer plasmid, either PRRL-SFFV LucII expressing Luciferase 2 transgene under control of the Spleen Focus Forming Virus (SFFV) promoter or pRRL-SFFV-LucII-2A-ΔNGFR-WPRE expressing Luciferase 2 transgene and a truncated form of the nerve growth receptor (NGFR) transgene in a bicistronic cassette under control of the Spleen Focus Forming Virus (SFFV) (22.5 μg). After 24 hours, the cells are washed and fresh medium is added. The following day, medium is harvested, clarified by centrifugation 1500 rpm for 5 min and filtered 0.45 μm, then concentrated by ultracentrifugation 50000 g for 2 h at 12° C. and stored at −80° C. until used. VSVg-pseudotype particles were produced also by transient transfection as reported (Merten et al, Human gene therapy, 2011, 22, 343-356).

c. Titration of Syncytin-A-Pseudoptyped LV

Physical titer was determined by p24 ELISA (Alliance© HIV-1 Elisa kit, Perkin-Elmer, Villebon/Yvette, France) followed by a calculation of the titer as physical particles (pp) assuming that 1 fg of p24 corresponds to 12 pp of LV (Farson et al, Hum Gene Ther. 2001, 20, 981-97), as previously reported for other types of LV (Charrier et al, Gene therapy, 2011, 18, 479-487). Infectious titer was determined as infectious genome titer (IG/mL) using the murine lymphoma cell line A20. Serial dilutions of vector are added to A20 cells in the presence of Vectofusin-1® (12 μg/μL) for 6 hours. Medium is renewed and cells are incubated for 7 days and genomic DNA is obtained to measure vector copy number per cells using duplex qPCR on iCycler 7900HT (Applied Biosystems) with the primers: PSI forward 5′CAGGACTCGGCTTGCTGAAG3′ (SEQ ID NO:7), PSI reverse 5′TCCCCCGCTTAATACTGACG3′ (SEQ ID NO:8), and a PSI probe labeled with FAM (6-carboxyfluoresceine) 5′CGCACGGCAAGAGGCGAGG3′ (SEQ ID NO:9), Titin forward 5′AAAACGAGCAGTGACGTGAGC3′ (SEQ ID NO:10), Titin reverse 5′TTCAGTCATGCTGCTAGCGC3′ (SEQ ID NO:11) and a Titin probe labeled with VIC 5′TGCACGGAAGCGTCTCGTCTCAGTC3′ (SEQ ID NO:12).

Results

Murine Syncytins were explored as possible new pseudotype for HIV-1-derived LV for in vivo applications. Syncytin A is non-orthologue but functionally similar murine counterpart to human Syncytins-1 and -2 (Dupressoir et al, Proceedings of the National Academy of Sciences of the United States of America, 2005, 102, 725-730).

The murine SynA was cloned into an expression plasmid and used to produce lentiviral vector particles in 293T cells. It was found that SyncytinA can successfully pseudotype rHIV-derived LV. An optimization of the amount of SyncytinA plasmid for the transfection step increased the production of LV particles based on p24 levels in medium. In the conditions defined (20 μg DNA per plate, one harvest only; see Materials and Methods), it was possible to produce stable and infectious particles pseudotyped with murine syncytin. Lentiviral particles pseudotyped with this envelope could be successfully concentrated by ultracentrifugation using the same conditions as used for VSVg-pseudotyped particles (Charrier et al, Gene therapy, 2011, 18, 479-487). The concentrated stocks were cryopreserved at −80° C. and were stable for several months. LV-Syn A was very efficient at transducing the murine A20 B lymphoma cell line in the presence of Vectofusin-1 (VF1). The A20 cell line is used to generate the infectious titer for Syncytin-A-pseudotyped LV.

### EXAMPLE 2

### In Vivo Gene Delivery to the Lung Using LV-SynA Particles

Materials and Methods

Animals

Male or female 6 week old C57/Bl6 albinos mice were injected with 100 μL of LV-SynA (equivalent to 3.10⁵ ng p24) or 100 μL of PBS for the control mice in the tail vein. Mice are analyzed by bioluminescence at different time points and are sacrificed by cervical elongation at day 21 post-injection. Lungs are removed after sacrifice. The right lung is used fresh to sort the cells and realize qPCR. The left lung is frozen in isopentane and conserved at −80° C. to perform cryostat slices and immunohistostaining.

In Vivo Luciferase Imaging

C57BL/6 mice were anesthetized with ketamine (120 mg/kg) and xylasine (6 mg/kg) and 100 μL (150 μg/mL) of D-luciferin (Interchim, ref FP-M1224D) was administered intra-peritoneally and imaged 10 min later with a CCD camera ISO14N4191 (IVIS Lumina, Xenogen, MA, USA). A 3 min bioluminescent image was obtained using 10 cm field-of-view, binning (resolution) factor 4, 1/f stop and open filter. Region of interest (ROIs) were defined manually (using a standard area in each case), signal intensities were calculated using the living image 3.2 software (Xenogen) and expressed as photons per second. Background photon flux was defined from an ROI drawn over the control mice in which no vector had been administered.

Lung Cell Sorting

Lung are perfused with collagenase IV (1 mg/mL, Invitrogen) and DNase I (50 μg/mL, Roche) and then incubated at 37° C. 45 min. The reaction is stopped by the addition of EDTA (100 mM). The cells are then isolated by dilaceration. Lung cells are stained with an anti-CD45-FITC antibody (BD Pharmingen, ref 553080) and an anti-CD31-BV510 antibody (BD Horizon, ref 563089). Cells are then sorted on the MoFlo® Astrios (Beckman Coulter).

qPCR

Genomic DNA is extracted from the cells using the Wizard® Genomic DNA Purification Kit (Promega, ref. A1125). The multiplex qPCR is performed either on the PSI proviral sequence or on the WPRE proviral sequence, with the TitinMex5 as a normalization gene. The following primers and probes are used at a concentration of 0.104:

| | |
|---|---|
| PSI F | 5′ CAGGACTCGGCTTGCTGAAG 3′ |
| | (SEQ ID NO: 7) |
| PSI R | 5′ TCCCCCGCTTAATACTGACG 5′ |
| | (SEQ ID NO: 8) |
| PSI probe (FAM) | 5′ CGCACGGCAAGAGGCGAGG 3′ |
| | (SEQ ID NO: 9) |
| WPRE F | 5′ GGCACTGACAATTCCGTGGT 3′ |
| | (SEQ ID NO: 13) |
| WPRE R | 5′ AGGGACGTAGCAGAAGGACG 3′ |
| | (SEQ ID NO: 14) |
| WPRE probe (FAM) | 5′ ACGTCCTTTCCATGGCTGCTCGC 3′ |
| | (SEQ ID NO: 15) |
| TitinMex5 F | 5′ AAAACGAGCAGTGACGTGAGC 3′ |
| | (SEQ ID NO: 10) |
| TitinMex5 R | 5′ TTCAGTCATGCTGCTAGCGC 3′ |
| | (SEQ ID NO: 11) |
| TitinMex5 | 5′ TGCACGGAAGCGTCTCGTCTCAGTC 3′ |
| probe (VIC) | (SEQ ID NO: 12) |

The qPCR mix used is ABsolute qPCR ROX mix (Thermo Scientific, ref CM-205/A). The analysis is performed on the iCycler 7900HT (Applied Biosystems) with the SDS 2.4 software.

PCR

A PCR using the Taq Phusion (Thermo Scientific, ref. F-5495) is performed on gDNA from the lungs. The following primers are used at a concentration of 0.1 μM:

(SEQ ID NO: 19)
Psi-F: 5′ AGCCTCAATAAAGCTTGCC 3′
(SEQ ID NO: 20)
RRE-R: 5′ TCTGATCCTGTCGTAAGGG 3′

The PCR program is 98° C. 30 s→(98° C. 10 s, 61° C. 30 s, 72° C. 45 s)×35→72° C. 5 min. The PCR product is placed on a 2% agarose gel for electrophoresis and the expected band is at 489 bp.

Immunohistostaining on Lung Sections

Cryostat sections of mice lung (12 μm) are fixed in 4% paraformaldehyde solution during 10 min and then washed 3 times in PBS 1×. Sections are then stained with a polyclonal antibody anti-luciferase (Promega, ref G7451) diluted at 1/100 as a primary antibody and a donkey anti-goat AlexaFluor 594 (Invitrogen, ref A11058) diluted at 1/1000 as a secondary antibody. The primary antibody is incubated overnight at 4° C. in a humidity chamber and the secondary antibody is incubated for 2 h in a humidity chamber.

Results

The objective was to determine the biodistribution of syncytin-A-pseudotyped LV following intravenous systemic delivery. The transgene luciferase was used because it is bioluminescent and enables dynamic detection over time. Two different LVs coding for Luc2 were tested in four different mouse in vivo protocols. One LV was encoding only Luc2 transgene (LV-SA-LucII), the other was encoding Luc2 and dNGFR in a bicistronic cassette (LV-SA-LucII2AdNGFR). The bicistronic vector is much less potent to express Luc2. The bicistronic vector is therefore useful to confirm the transduction of organs by qPCR but the expression of transgene by bioluminescence was not optimal and therefore not quantified. As control, a LV-VSVG Luc2 was used. Four different in vivo protocols in mice were done to inject the vectors and measure transduction over time. The dose of vector is the maximal dose that can be used in a 100 μL volume of injection and corresponds to about 3×10⁵ ng p24 or 5×10⁵ IG per mouse. Transgene expression was measured in the mice at different time points (1, 2 and 3 weeks post injection) by in vivo bioluminescence detection and to confirm transgene expression with a different technique, luciferase immuno-histochemistry detection was performed on some mice 3 weeks after injection. Transduction was measured by qPCR 3 weeks post injection.

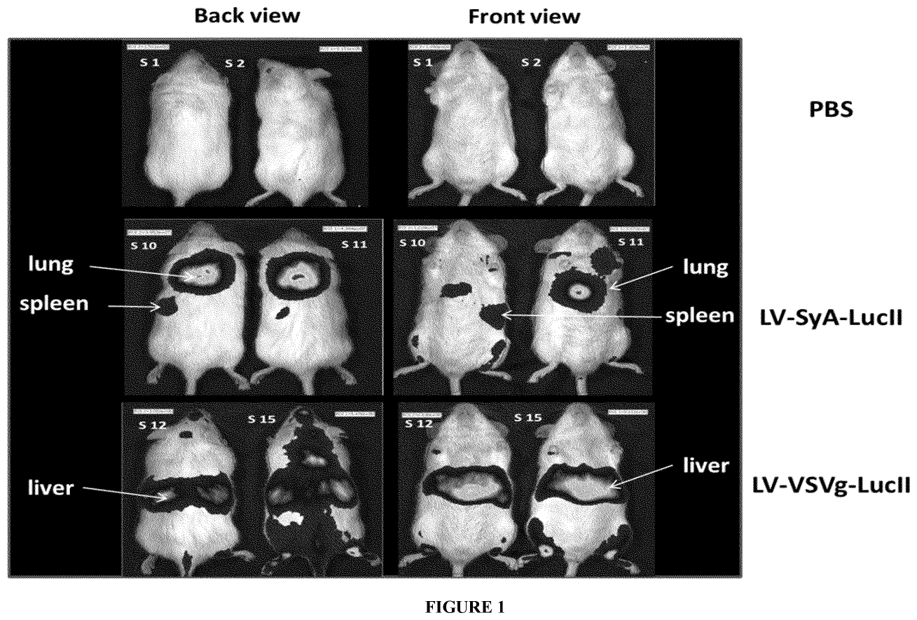

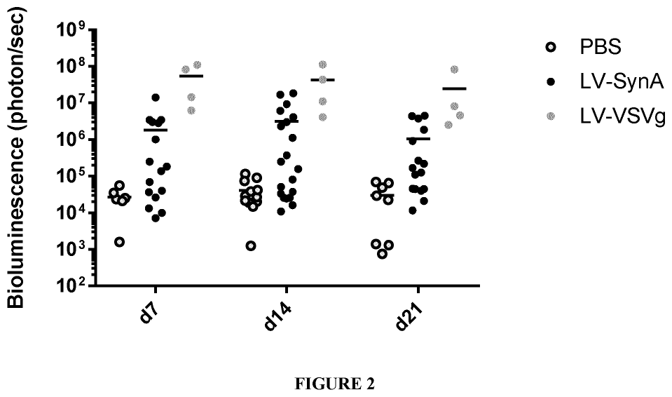

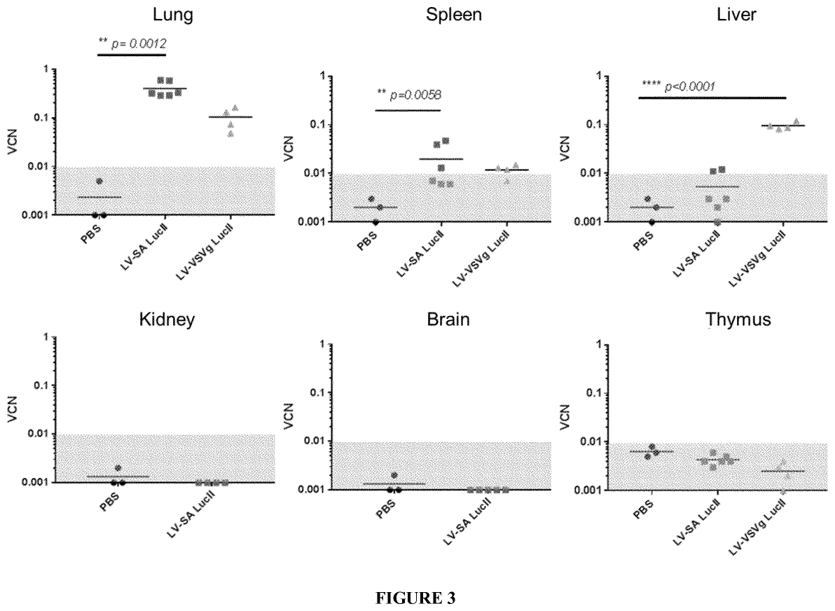

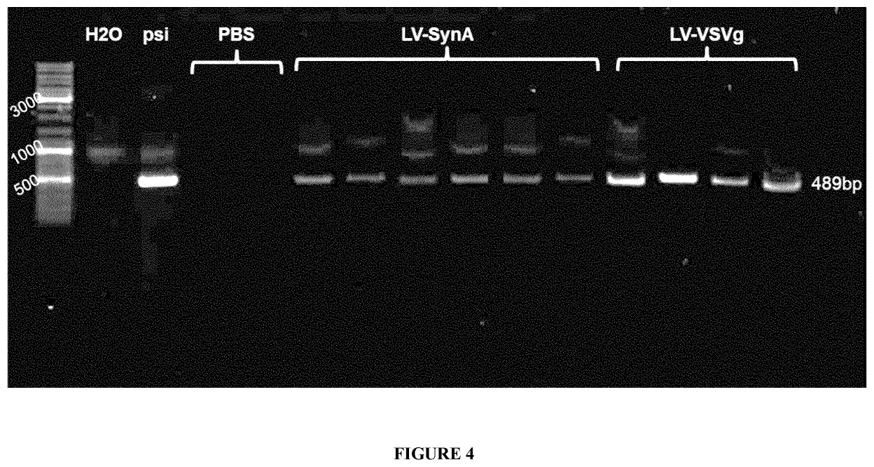

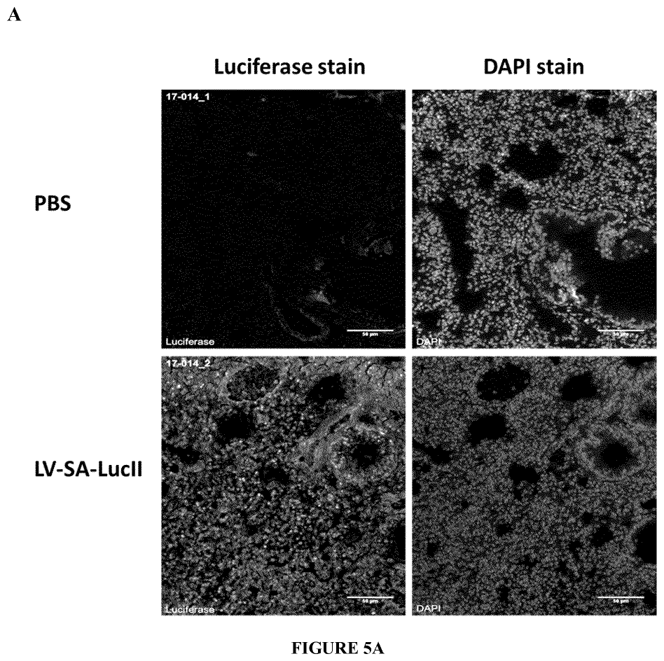

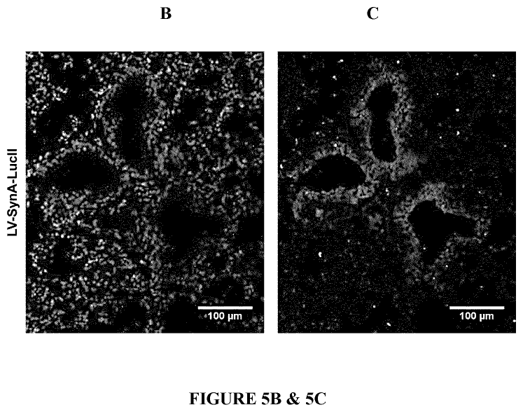

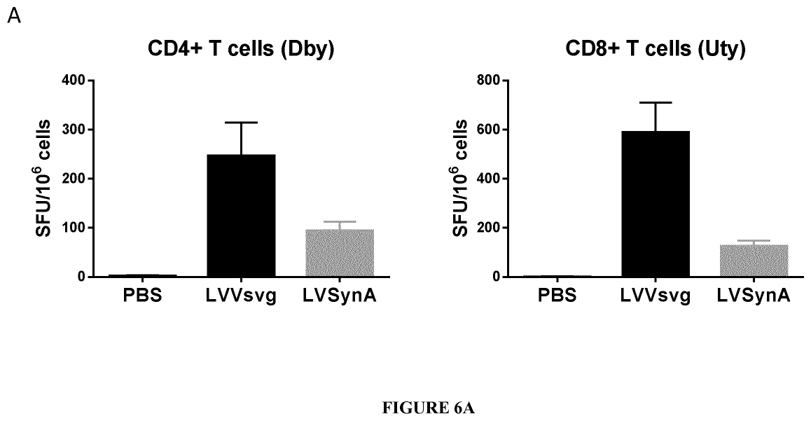

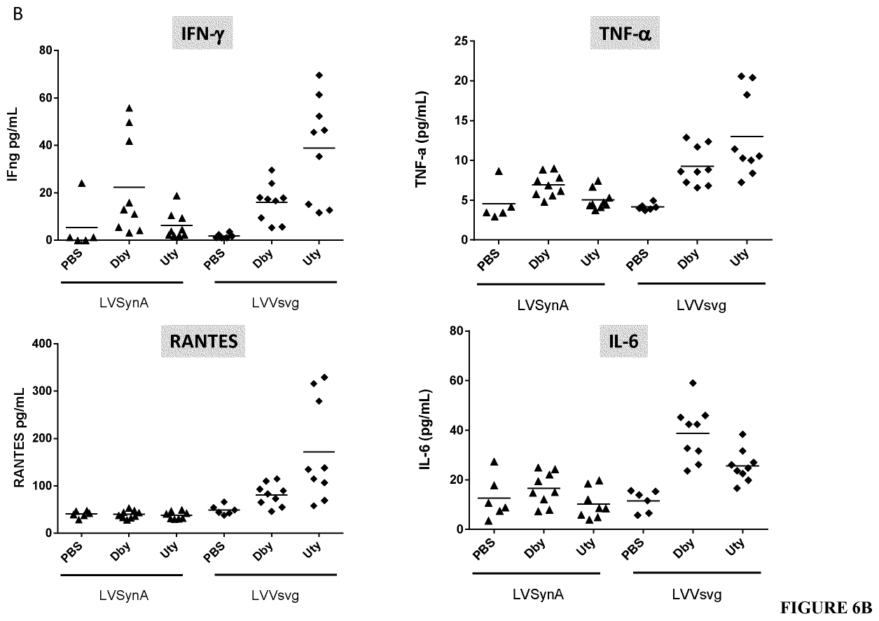

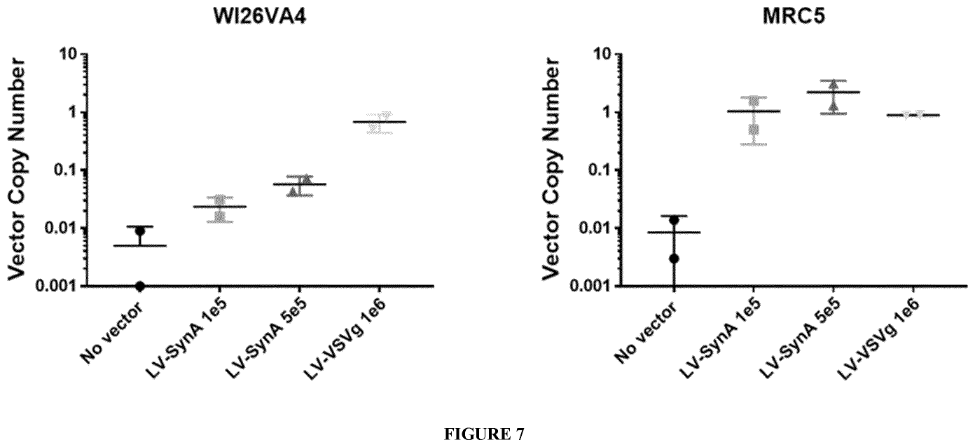

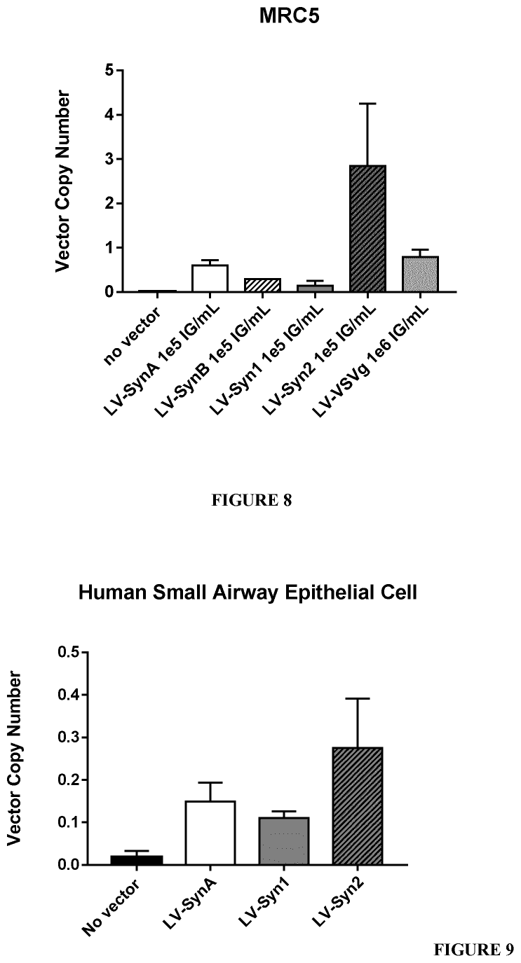

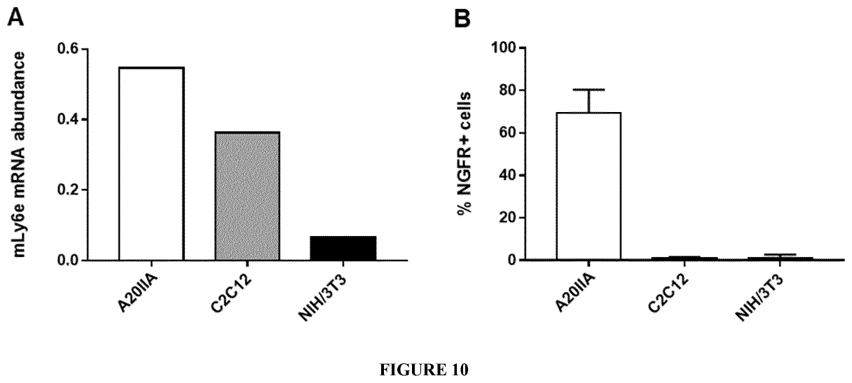

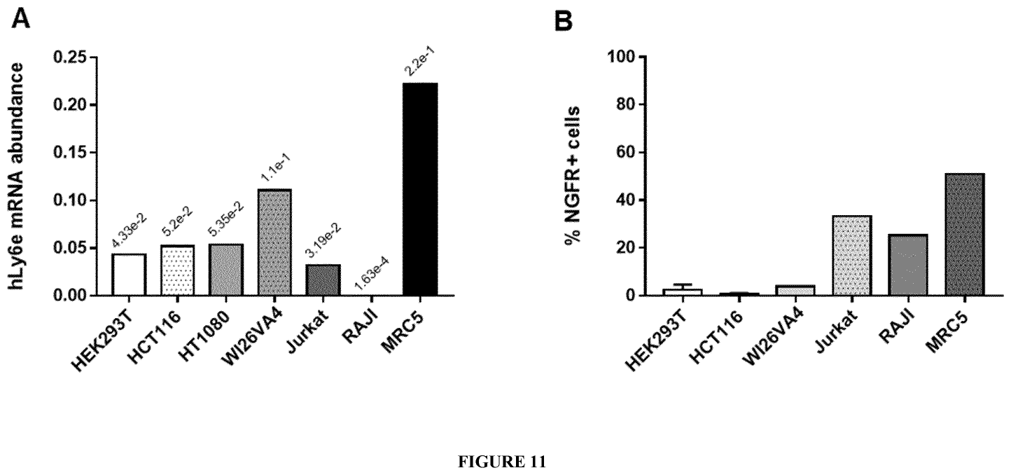

FIG. 1 shows the bioluminescence analysis in representative mice at week 2. A clear signal is observed in spleen and in the lungs following syncytin A LV delivery. Contrary to VSVG, syncytin A does not transduce liver.

The quantification of the lung signal was done in individual mice over time (upon exposure of the back). The signal obtained with LV-SA is strong and persisted over time as shown in FIG. 2B. These results show that a single intravenous administration of a LV-SynA vector to mice provides a significant, stable and long-lasting gene transfer in the lung, as detected with a bioluminescent transgene.

The amount of vector was measured by qPCR in the different organs recovered at the time of sacrifice of the mice at week 3. Results showed that vector copies were found preferentially in lung and spleen when administered by syncytin A-LV and in lung and liver when administered by VSVG-LV (FIG. 3). PCR around the PSI sequence of the integrated provirus confirms the detection of the transgene cassette in the lung of mice, 3 weeks after a single intravenous injection of LV-SynA vector, suggesting that stable integrative gene transfer can be achieved (FIG. 4).

Overall, by measuring transduction and transgene expression in lung, spleen and liver, it is clear that Syncytin-A-LV has a unique transduction profile. Based on qPCR, SyncytinA-LV can transduce the lung and spleen very efficiently but not the liver. The bioluminescent signal confirms the transduction of lung and spleen by Syncytin-A. While an average signal is obtained in the liver area, probably partially due to the adjacent signal in spleen, the levels are much weaker compared to that obtained with VSVg (Table I). VSVg-pseudotyped LV transduce liver very efficiently as shown by qPCR and bioluminescence.

TABLE I
Different tropism of syncytin-A- and VSVg-pseudotyped LV

| A | Average Vector Copy Number per Cell in organ ± SD (n) | | |
|---|---|---|---|
| Group | Lung | Spleen | Liver |
| PBS | 0.00 ± 0.00 (n = 7) | 0.00 ± 0.00 (n = 8) | 0.00 ± 0.00 (n = 6) |
| LV-SA LucII | 0.17 ± 0.17 (n = 8) | 0.01 ± 0.01 (n = 8) | 0.00 ± 0.00 (n = 5) |
| LV-SA | 0.32 ± 0.19 (n = 5) | 0.03 ± 0.02 (n = 9) | 0.00 ± 0.01 (n = 9) |
| LucIIdNGFR | | | |
| LV-VSVg LucII | 0.19 ± 0.06 (n = 4) | 0.02 ± 0.01 (n = 4) | 0.08 ± 0.05 (n = 4) |

| B | Average Bioluminescence of organ (photons/sec) × E+04 +/− SD (n) | | |
|---|---|---|---|
| Group | Lung | Spleen | Liver |
| PBS | 2 ± 1 (n = 8) | 2 ± 0 (n = 8) | 7 ± 2 (n = 8) |
| LV-SA LucII | 647 ± 1010 (n = 9) | 214 ± 229 (n = 9) | 233 ± 219 (n = 9) |
| LV-VSVg LucII | 6160 ± 7310 (n = 4) | 4680 ± 2840 (n = 4) | 42500 ± 28700 (n = 4) |

Table I (A-B) legend: Transduction levels and expression of the bioluminescent transgene luciferase were quantified in lung, spleen and live. Average values, SD and number of mice tested (n) were obtained in 4 different protocols (ranging from 1 to 4 depending on vector tested). A. Transduction was measured by qPCR 3 weeks after injection of vector. B. Bioluminescence was measured 2 weeks after injection of vector. Quantification was done by drawing a mask to define the organ area based on the largest area detected by the highest signal. The same mask was applied to all mice from a same protocol. The signal for lung was measured on the back of the mice. The signals for spleen and liver were measured on the front of the mice. The bioluminescent signal obtained with the bicistronic LV-SA LucII-dNGFR vector being much weaker than LV-SA LucII was not indicated.

The transduction of lung cells was examined in greater detail. Lung is a complex tissue containing alveoli composed of a single layer of squamous epithelial cells. Alveoli are separated from one another by connective tissue, interlaced with numerous capillaries and with infiltrating cells such as macrophages. The presence of the transgene Luc2 (LucII) was demonstrated to be in lung epithelial cells by 2 techniques. First, lungs were digested with a mixture of collagenase DNAse and the cells were stained with CD45 antibodies to recognize and sort CD45+ cells of hematopoietic origin and CD45− cells of non-hematopoietic origin i.e. lung parenchyma or stroma. The sorted cell DNA was extracted and analyzed by qPCR. Results show the presence of vector copies only in CD45− lung cells and not in the hematopoietic fraction (Table II). The level of transduction is coherent with the broad and clear bioluminescence signal observed.

TABLE II
Transduction of lung stromal cells

| | mouse | cells | VCN |
|---|---|---|---|
| PBS | 1 | Total | 0 |
| LV-SA-LucII | 2 | CD45+ | 0 |
| | | CD45− | 0.45 |
| | 3 | total | 0.01 |

Immunohistochemistry staining of Luc2 was performed on frozen lung sections. Results suggest that Luc2 was expressed in epithelial cells of the lung throughout the organ (FIG. 5). A staining done on paraffin-embedded lung showed that the epithelial cells lining the alveoles are expressing the transgene (FIG. 5B) In some experiments, a double staining of F4/80 macrophages was done and did not show any Luc2 in macrophages, thus confirming the qPCR results. In addition, a staining of CD31+ lung endothelium was done and did not correspond to the marking obtained with Luc2.

The results show that the biodistribution of syncytin-A-LV is very different from that of VSVg-LV. Contrary to VSVg, syncytin A does not transduce liver and instead, transduced at high levels the mouse spleen and lungs. Thus, syncytin A LV could be useful as drug and gene delivery tools for lung epithelium including for lung gene therapy.

### EXAMPLE 3

### Reduced Immune Response Against Transgene Following Systemic Delivery Using LV-SynA, Compared to LV-VSVg

Materials and Methods

Determination of the Immune Response by ELISPOT

IFN-γ enzyme-linked immunospot assays (ELISPOT) were performed by culturing 10⁶ spleen cells per well with or without 1 μM of Dby or Uty peptide in IFN-γ Enzyme-Linked Immunospot plates (MAHAS45, Millipore, Molsheim, France). As a positive control, cells were stimulated with Concanavalin A (Sigma, Lyon, France) (5 μg/ml). After 24 h of culture at +37° C., plates were washed and the secretion of IFNγ was revealed with a biotinylated anti-IFNγ anti-body (eBiosciences), Streptavidin-Alkaline Phosphatase (Roche Diagnostics, Mannheim, Germany), and BCIP/NBT (Mabtech, Les Ulis, France). Spots were counted using an AID reader (Cepheid Benelux, Louven, Belgium) and the AID ELISpot Reader v6.0 software. Spot forming units (SFU) are represented after subtraction of background values obtained with non-pulsed splenocytes.

Cytokine Titration by Cytometric Bead Array

Stimulation media [medium, Uty (2 μg/mL), Dby (2 μg/mL), or Concanavalin A (5 μg/mL)] were plated and 10⁶ splenocytes/well were added. After 36 h of culture at +37° C., supernatants were frozen at −80° C. until the titration. Cytometric bead arrays were performed with BD Biosciences flex kits (IL-6, IFN-γ, TNFα, and RANTES). Briefly, capture bead populations with distinct fluorescence intensities and coated with cytokine-specific capture antibodies were mixed together. Next, 25 μL of the bead mix of beads was distributed and 25 μL of each sample (supernatants) was added. After 1 h of incubation at room temperature, cytokine-specific PE-antibodies were mixed together and 25 μL of this mix was added. After 1 h of incubation at room temperature, beads were washed with 1 mL of Wash buffer and data were acquired with an LSRII flow cytometer (BD Biosciences). FCAP software (BD Biosciences) was used for the analysis.

Results

The reduced immunogenicity of LV-SynA after systemic administration was tested in comparative assays with LV pseudotyped with VSVg (LV-VSVg). In these studies the transgene used was GFP-HY which encodes a fusion protein consisting of GFP tagged with the male HY gene sequences. When the transgene is presented by antigen-presenting cells, the Dby and Uty peptides are presented to CD4 and CD8 T cells which enable the detection of transgene-specific immune responses. The results show that systemic, intravenous (IV) administration of LV-SynA vector encoding GFP-HY to mice leads to less and very low levels of anti-transgene CD4 and CD8 T cell immune responses (FIG. 6A) and lower levels of cytokines (FIG. 6B) compared to LV-VSVg. These results are coherent with the possibility to achieve long-term expression of a transgene in lung following gene delivery with syncytin-pseudotyped vectors. The results also suggest that repeated administrations of transgene can be achieved with these vectors. The results also suggest that syncytin-pseudotyped vectors can be used safely in inflammatory conditions, without inducing high levels of additional immune responses or inflammation.

### EXAMPLE 4

### Transduction of Human and Murine Lung Cell Lines with LV-Syncytins

A first objective of this study was to evaluate if human lung cells could be transduced with a lentiviral vector pseudotyped with human or murine Syncytins (Syncytin-A, -B, -1 or -2). A second objective of this study was to determine whether or not the transduction of different human and murine cell lines and murine primary cells with a lentiviral vector pseudotyped with Syncytin A is correlated with Ly6e expression on the target cells.

Materials and Methods

Human Primary Small Airway Epithelial Cells and Human Lung Cell Lines Transduction with LV-Syn

Lentiviral vectors coding for Luc2 or ΔNGFR and pseudotyped with Syn-A, -B, -1 or -2 were used for these experiments. The vectors were tested by culturing at 37° C. 1×10⁵ MRC5 cells (human lung fetal cells, ECACC, ref 84101801), WI26VA4 cells (SV40-transformed human lung cells, ATCC, ref CCL-95.1) or Human Small Airway Epithelial Cells (Primary Small Airway Epithelial Cells; Normal, Human (ATCC® PCS301010™) with one concentration or two concentrations of LV-SynA lentiviral particles (1×10⁵ or 1×10⁵ and 5×10⁵ infectious genome (IG)/mL (infectious genome units defined on A20 cells)) in the presence of 12 μg/mL of Vectofusin-1® (Miltenyi Biotec, ref 130-111-163). As a positive control, cells were also cultured in parallel with 1×10⁶ IG (infectious genome units defined on HCT116 cells/mL) of LV-VSVg in the presence of Vectofusin-1. After 6 h of transduction at 37° C., the infection was stopped by changing the culture medium and adding fresh medium (DMEM+10% fetal calf serum+1% penicillin-streptomycin+1% glutamine) to the cells.

Three or seven days post-transduction, genomic DNA of the cells was extracted using the Wizard® Genomic DNA Purification Kit (Promega, ref A1125). Multiplex qPCR was performed to determine the vector copy number per cell using amplification of the PSI proviral sequence and albumin as a normalization gene. The following primers and probes were used at a concentration of 0.1 μM:

| | |
|---|---|
| PSI F | 5′ CAGGACTCGGCTTGCTGAAG 3′ |
| | (SEQ ID NO: 7) |
| PSI R | 5′ TCCCCCGCTTAATACTGACG 3′ |
| | (SEQ ID NO: 8) |
| PSI probe (FAM) | 5′ CGCACGGCAAGAGGCGAGG 3′ |
| | (SEQ ID NO: 9) |
| Albumin F | 5′ GCTGTCATCTCTTGTGGGCTGT 3′ |
| | (SEQ ID NO: 16) |
| Albumin R | 5′ ACTCATGGGAGCTGCTGGTTC 3′ |
| | (SEQ ID NO: 17) |
| Albumin probe | 5′ CCTGTCATGCCCACACAAATCTCTCC 3′ |
| (VIC) | (SEQ ID NO: 18) |

The qPCR mix used was ABsolute qPCR ROX mix (Thermo Scientific, ref CM-205/A). The analysis was performed on the iCycler 7900HT (Applied Biosystems) with the SDS 2.4 software or on the LightCycler480 (Roche) with the LightCycler® 480 SW 1.5.1 software.

Ly6e mRNA Expression on Different Human and Murine Cell Lines and Murine Primary Cells.

mRNA from different human cell lines (HEK293T, HCT116, HT1080, WI26VA4, Jurkat and RAJI), murine cell lines (A20IIA, C2C12, NIH/3T3) and from total cells from the lung, spleen and bone marrow of C57BL/6 mice were extracted using the RNeasy® mini kit from Qiagen. The reverse transcription of the mRNA was performed using Verso cDNA synthesis kit from Thermofischer. A qPCR was performed on the cDNA using the following primers: mLy6e forward primer 5′ CGGGCTTTGGGAATGTCAAC 3′ (SEQ ID NO: 21), mLy6e reverse primer 5′ GTGGGATACTGGCACGAAGT 3′ (SEQ ID NO: 22), hLy6e forward primer 5′ AGACCTGTTCCC CGGCC 3′ (SEQ ID NO: 23), hLy6e reverse primer 5′ CAGCTGATGCCCATGGAAG 3′ (SEQ ID NO: 24), PO reverse primer 5′ CTCCAAGCAGATGCAGCAGA 3′ (SEQ ID NO: 25) and PO forward primer 5′ ACCATGATGCGCAAGGCCAT 3′ (SEQ ID NO: 26). PO was used as a warehouse gene. The abundance is calculated with the formula abundance=2-ΔCt.

Results

Human primary small airway epithelial cells and human lung cell lines transduction with LV-Syn(-A, -B, -1, -2)

The level of transduction of MRC5 and WI26VA4 cells following infection with a lentiviral vector pseudotyped with the murine syncytin A (LV-SynA) was measured in two independent transduction experiments. The level of transduction of MRC5 cells following infection with a lentiviral vector pseudotyped with the murine syncytin A (LV-SynA), the murine syncytin B (LV-SynB), the human syncytin 1 (LV-Syn1) or the human syncytin 2 (LV-Syn2) was also measured in two or three independent transduction experiments. A control lentiviral vector pseudotyped with VSVg (LV-VSVg) and used at a high concentration confirmed that the cells could be transduced in the experimental conditions used.

In addition, the level of transduction of Human Small Airway Epithelial cells following infection with a lentiviral vector pseudotyped with the murine syncytin A (LV-SynA), the human syncytin 1 (LV-Syn1) or the human syncytin 2 (LV-Syn2) was measured in five (SynA) or three (Syn1, Syn2) independent transduction experiments.

In all experiments, the integration of the provirus in the cells was confirmed with a qPCR using specific primers. The results are presented in FIGS. 7 to 9.

FIG. 7 represents the average levels of transduction of the MRC5 and WI26VA4 cells with a lentiviral vector pseudotyped with the murine syncytin A (LV-SynA) in 2 experiments. Two concentrations of vector were used (1 E+05 and 5E+05 ig/mL) showing a dose-dependent effect. Clearly, the transduction of MRC5 cells with the LV-SynA vector was more efficient than the transduction of WI26V4 cells, but both cell types were permissive. Vectors pseudotyped with VSVg were used at a higher concentration, as positive controls. In conclusion, the syncytin A pseudotype can be used to transduce human lung cells.

FIG. 8 shows that in addition to murine SynA, the human Syn2 can also transduce human lung cells, thereby supporting the use of Syncytin2-pseudotyped lentiviral vectors for therapeutic applications in lung. The Syncytin-2 pseudotype is very effective as it reaches levels at least as high as those of the positive VSvg control which was used at 10× higher concentration.

FIG. 9 shows that the murine syncytin A and the human syncytin 2 can be used to pseudotype lentiviral vectors to efficiently transduce human primary pulmonary epithelial cells. These results further demonstrate that syncytin-pseudotyped particles can be used to treat pulmonary diseases and in particular, diseases involving the lung epithelium and that human syncytin-pseudotyped vectors would be expected to deliver transgenes in lung following systemic administration.

Comparison Between the Level of Expression of Ly6e mRNA and the Level of Transduction in Different Cell Lines.

The level of expression of mLy6e and hLy6e mRNA and the level of transduction with LV-Syncytin A vectors encoding ΔNGFR were compared in different cell lines.

The results show that the expression of mLy6e, reported as the receptor for murine Syncytin A, on cell lines does not allow to predict the ability to transduce cells by LV pseudotyped with SynA (FIG. 10). C2C12 cells express relatively abundant levels of Ly6e but are not transduced. A20IIA cells which express the highest levels of Ly6emRNA are transduced, which may indicate that a threshold exists.

The receptor for mouse SynA is mouse Ly6e. It is not known if the human Ly6e is a receptor for any of the syncytins. The results show that there is no correlation between human Ly6e receptor expression mRNA levels and transduction with LV-SynA (FIG. 11). HCT116 which are colon carcinoma cells express hLy6e mRNA but are not transduced. Raji cells do not express the human Ly6e-mRNA but can be transduced.

1-15. (canceled)

16. A method of preventing and/or treating lung diseases in a patient in need thereof, comprising administering to the patient a therapeutically effective amount of a pharmaceutical composition targeting lung tissue, comprising at least a therapeutic drug associated to a syncytin protein.

17. The method according to claim 16, wherein the syncytin protein is human or murine syncytin.

18. The method according to claim 17, wherein the syncytin is selected from the group consisting of human Syncytin-1, human Syncytin-2, murine syncytin-A and murine syncytin-B.

19. The method according to claim 16, wherein the therapeutic drug and the syncytin protein are incorporated into particles.

20. The method according to claim 19, wherein the particles are selected from the group consisting of liposomes, exosomes, viral particles and virus-like particles.

21. The method according to claim 19, wherein the syncytin protein is displayed on the surface of the particles.

22. The method according to claim 19, wherein the particles are lentiviral or lentiviral-like particles pseudotyped with syncytin protein.

23. The method according to claim 16, wherein the drug is selected from the group consisting of therapeutic genes, genes encoding therapeutic proteins or peptides, therapeutic antibodies or antibody fragments, genome editing enzymes, interfering RNA, guide RNA for genome editing, antisense RNA capable of exon skipping; anti-bacterial drugs, anti-viral drugs, anti-fungal drugs, anti-parasitic drugs; anti-inflammatory drugs; immunotherapeutic drugs, immunomodulatory drugs, immunosuppressive drugs, anti-allergic drugs, anti-histaminic drugs and immunostimulating drugs.

24. The method according to claim 19, wherein the drug is a gene of interest packaged into a viral vector particle.

25. The method according to claim 16, wherein the drug is a gene of interest packaged into a lentiviral vector particle pseudotyped with syncytin protein.

26. The method according to claim 16, wherein the lung diseases are selected from the group consisting of: genetic diseases affecting the lungs; infectious diseases affecting the lungs; inflammatory or auto-immune diseases of the lungs, asthma, chronic obstructive pulmonary disease, pulmonary fibrosis, oedema, emphysema, hypertension, acute respiratory distress syndrome, pneumoconiosis, interstitial lung disease, diffuse parenchymal lung disease, lung transplant rejection and lung disease in new born and premature babies.

27. The method according to claim 16, for use in gene therapy of the lung diseases.

28. The method according to claim 16, wherein the drug is a gene of interest for therapy selected from the group consisting of: SERPINA3, SERPINA1, MMP, in particular MMP1, MMP2 and MMP9, CFTR, SFTPB, SFTPC, ABCA3, CSF2RA, TERT, TERC, SFTPA2, SLC34A2, DKC1, TERC, TERT, TINF2, NF1, TSC1, FLCN, STAT3, HPS1, GBA, SMPD1, SLC7A7, SMAD9, KCNK3 and CAV1 genes, and functional variants thereof.

29. The method according to claim 16, which is for administration by injection, inhalation or broncho-alveolar lavage.

30. A pharmaceutical composition targeting lung tissue, comprising virus particles pseudotyped with syncytin protein, packaging a gene of interest selected from the group comprising: the genes SERPINA3, SERPINA1, MMP, in particular MMP1, MMP2 and MMP9, CFTR, SFTPB, SFTPC, ABCA3, CSF2RA, TERT, TERC, SFTPA2, SLC34A2, DKC1, TERC, TERT, TINF2, NF1, TSC1, FLCN, STAT3, HPS1, GBA, SMPD1, SLC7A7, SMAD9, KCNK3, CAV1, functional variants thereof, interfering RNA, guide RNA for genome editing, antisense RNA capable of exon skipping, wherein the RNA target the gene of interest.

31. The pharmaceutical composition according to claim 30, wherein the virus particles are lentiviral vector particles.

32. A pharmaceutical composition for targeting lung tissue, comprising virus-like particles, pseudotyped with syncytin protein, an interfering RNA, guide RNA for genome editing or antisense RNA capable of exon skipping, said RNA targeting a gene of interest selected from the group of genes comprising: SERPINA3, SERPINA1, MMP, in particular MMP1, MMP2 and MMP9, CFTR, SFTPB, SFTPC, ABCA3, CSF2RA, TERT, TERC, SFTPA2, SLC34A2, DKC1, TERC, TERT, TINF2, NF1, TSC1, FLCN, STAT3, HPS1, GBA, SMPD1, SLC7A7, SMAD9, KCNK3 and CAV1.

33. The pharmaceutical composition according to claim 32, wherein the virus-like particles are lentivirus-like particles.


SEQUENCE LISTING (26 sequences)

SEQ ID NO: 1 - DNA, 1617 bp, Artificial Sequence
  synthetic polynucleotide (cDNA of human HERV-W)
atggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttag

SEQ ID NO: 2 - DNA, 1617 bp, Artificial Sequence
  synthetic polynucleotide (cDNA of human HERV-FRD)
atgggcctgctcctgctggttctcattctcacgccttcactagcagcctaccgccatcctgatttcccgttattggaaaaagctcagcaactgctccaaagtacaggatccccttactccaccaattgctggttatgtactagctcttccactgaaacaccagggacagcttatccagcctcgcccagagaatggacaagcatagaggcggaattacatatttcctatcgatgggaccctaatctgaaaggactgatgaggcctgcaaatagtcttctttcaacagtaaagcaagatttccctgatatccgccagaaacctcccattttcggacccatctttactaatatcaacctaatgggaatagcccctatttgtgttatggccaaaaggaaaaatggaacaaatgtaggcactcttccaagtacagtctgtaatgttactttcactgtagattctaaccaacagacttaccaaacatacacccacaaccaattccgccatcaaccaagattccccaaacctccaaatattacttttcctcagggaactttgctagataaatccagccggttttgccagggacgcccaagctcatgcagtactcgaaacttctggttccggcctgctgattataaccaatgtctgcaaatttccaacctcagctctacagcggaatgggttctattggaccaaactcgaaattctcttttttgggaaaataaaaccaagggagctaaccagagccaaacaccctgcgtccaagtcttagcaggcatgactatagccaccagctacctgggcatatcagcagtctcagaattttttggaacctccctcacccccttatttcatttccatatctctacatgccttaaaactcaaggagccttttatatttgtggccagtcgattcaccaatgcctccccagtaactggactggaacttgtaccataggctatgtaaccccagacatcttcatagcccctggcaatctctctcttccaataccaatctatgggaattccccgttgcccagggtgaggagggcaatccatttcattccccttctcgcgggactcggcattctagctggtacgggaaccggaattgctggaatcacaaaagcttccctcacctatagccagctctcaaaggaaatagccaacaacattgacaccatggctaaagccttaacgaccatgcaagaacaaatcgactctttagcagccgtagtccttcaaaatcgtcgaggactagacatgttaacggcagcacagggaggaatttgtttggccttagatgaaaaatgttgcttttgggtaaatcaatcaggaaaagtacaagacaacatcagacaactcctaaatcaagcctccagtttacgggaacgagccactcagggttggttaaattgggaaggaacttggaaatggttctcttgggttcttccccttacaggcccacttgttagtctcctacttttgctcctttttggtccatgtctcctaaatctaataacccaatttgtctcctctcgccttcaggccataaagctccagacgaatctcagtgcaggacgccatcctcgcaatattcaagagtcacccttctaa

SEQ ID NO: 3 - DNA, 1854 bp, Artificial Sequence
  synthetic polynucleotide (cDNA murine Syncytin A )
atggttcgtccttgggttttctgtctcctcctgttcccttgttcctctgcctactcggacagctggatgcccctggtaaacctcactcaacacctcctccaggaggctaactcttccttctcttccaactgctgggtctgcttatccatccaaacccagcgctctctagccatgccagccccactaaggacttggacagagacacccatgaaacttcgaatcatgtactcagcccggaccctctccggcccataccctatcaccgaccttgagaggcgcctccagaatttccaaccattgactccccactcctcttttgtcaaccctgaccagcgggccattgctttccttcagatcaccagcgtgacaggcatacttcccatactttctcggatcacctcggtgagataccccgatgaccacgtctatgaatctgcccagcgccccatatggggctcactctccacccagacgatcctcacctcccaggcccctctctgcatatcccgcttcttcaagaattcaaaccatgccaccttcgtgggcaaactccctgcctctctttgcaatcacacctttcagctctccccctctgccaaccaccaatccatagatctgtcctccagctatgcattcgccccattaatggccatgccagggtctaaatggagaaaccccttacgcttttcaggacccccttccctgaactcagggatgcctcactactcctgcccgatagatgacatccactgccacacctaccccaccaccccctggaggtcctgtccttccttcccagctagcacctgctataatctcaccctattcgagccggacaattcgagccaccctattaccctgtctgtggacaccacatacttcaagattaaactccagggacacaaagacccctatccactctttcagtaccagcccctcatgggggcagccctctctggacaatattcaatctgggaatatgaacccactgttaagaaaaacggcggtatcactccaaatatcttctcccaccttgtctccttaacatactccttctgcctcaactcctctggtgttttcttcctctgtggaaactcaacttatgtctgtctcccggccaattggtccggcgtctgtacccttgtcttccaatacccggatattgaactccttcccaacaaccaaaccatatctgtcccgctttttgctacagttccctcctctgtccccgcttctcgccggaagcgagcccttcctctccttcctctcctcgcgggcctgggcattgcttctgccctggggttaggcatcgcgggtatcaccacctcaactgtgtatttccaacagctttccaaggctctctcggacagcctagatgaaatagccacctccatcatcagcctccaagaccaaatagactcgctggcgggtgtcgttctccaaaaccgcagagctctggacctcattgtggctgagagggggggcacctgcctcttcctccaggaagagtgctgcttctacataaaccagtccggggtagtccggcacgcggcaaggaaacttcgagaaagggcctcggaactcggcacaagctcgagttcttggatccagtggctggggctaggaccctggctgccctcttggttgacttccctcatggctcccattctctttatcctggtactgctggttttcaggccttgtcttcttaactgcctgactcattctgtatcgcggcgaatgagttctttcattcacaccaccaccgaaggacacgtggacaagatccttctgcttcgagagtcccagtacaagagacttccccaagagcccccggaggaggatgccgtctag

SEQ ID NO: 4 - DNA, 1857 bp, Artificial Sequence
  synthetic polynucleotide (cDNA murine Syncytin B)
atgacaggcttttgggtcctctgtttcgtccttttcccctcctccttatcctatccggaaagctggatgccccttgtaaacctcactcaccacatcctacgtgataccaactcttccctgttttccaactgttgggtctgcttgtctacccaaacccagcggtccttagcagtcccagcccctctgtccatttggacagatacacccatgaagcttcatcttacctactcagtcaggcccttctctggctccttttccattagcgacattgaaagacgcctccgtctcttccgcccactgactgcctcctattctttccacaatcctgacagaagggcgattgcttttcttcaactcgtcagctcaacaggcatatttcggatcatcacccggataacctctgtgatatatccccataaggaccgtttcttcgaatctgcccaacgccctctctggggaccactctttactgagaccgtgctcaggtcgcaggccccactctgcatatctcgctttttcaaggtctcagcatatgccacttttgtaggcaacctctctgcctctctctgcaactacaccatgcatatttcaccttctaccagtcatgaaaacctagatctttccaccacccatacgttcaaacaggcaatgaaaagaccggatgccaaatggaaaaacccgctccgtttttccgggcccccctccctcatcttctcgaagccggcttactatccctgcccaacagacatcaaacactgccatacctctccggccactccctggatgcactgtcctcaggctcccttcggcacctgctataacctcactttatttgaaccagacaactcaacccaccctgttaccatgtcagtgaaccctacccacttcaaggtcaaactccaggggcacagagacccctatccgctctcccattaccagcccctcacgggagctgccctgtctggacaatattcagtctgggagaacgagatcactgtccaagaaaactgggacatcacctccaacattttctcacatcttctcagcttctcgtacgccttctgcctcaactcttcaggcgttttcttcctctgcggaacatcgacttacatctgcctcccagccaattggtccggtgtctgtaccctggtcttccaatacccggatattgaacttctccccaataaccaaacggtgcctgttcccctttttgcttcagttctttcctcagactcagttcttcgcccaaagaggtcccctcacctctttcccttccttgcaggcctgggtatctcttctgcccttggtacggggatagctggcttggccacctcgactctctatttccaacagctttctaaggttctttccgaaaccttggaagaaatagctgcctctatcactaccctccagaaccaaatagactcgctcgcaggtgttgttctacaaaaccgccgagctctggacctcatcactgctgagaaagggggcacctgtctcttcctccaggaagagtgctgcttctacgtaaaccagtctggaatagtccgggacgcggcaaggaaactccaagaacgagcatctgaactcggccagcattctgactcttggggacagtggcctgaccttggacgttggttgccctggctgactccctttctgggacctcttctcttcctcttcttcctactgacatttgggtcttgtcttctgaactgcctaacccgttttgtgtcccagagacttggctcctttgttcaagacactgccaaaaggcatgtggacagcatcctccaaaatttccaatataaaaaactgccccaagactccccagatgaggacaccattcctacataa

SEQ ID NO: 5 - DNA, 7150 bp, Artificial Sequence
  synthetic polynucleotide (pcDNA3.1 SYNCYTIN-1)
tcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagc

SEQ ID NO: 6 - DNA, 7134 bp, Artificial Sequence
  synthetic polynucleotide (pcDNA3.1 SYNCYTIN-2)
cactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgggcctgctcctgctggttctcattctcacgccttcactagcagcctaccgccatcctgatttcccgttattggaaaaagctcagcaactgctccaaagtacaggatccccttactccaccaattgctggttatgtactagctcttccactgaaacaccagggacagcttatccagcctcgcccagagaatggacaagcatagaggcggaattacatatttcctatcgatgggaccctaatctgaaaggactgatgaggcctgcaaatagtcttctttcaacagtaaagcaagatttccctgatatccgccagaaacctcccattttcggacccatctttactaatatcaacctaatgggaatagcccctatttgtgttatggccaaaaggaaaaatggaacaaatgtaggcactcttccaagtacagtctgtaatgttactttcactgtagattctaaccaacagacttaccaaacatacacccacaaccaattccgccatcaaccaagattccccaaacctccaaatattacttttcctcagggaactttgctagataaatccagccggttttgccagggacgcccaagctcatgcagtactcgaaacttctggttccggcctgctgattataaccaatgtctgcaaatttccaacctcagctctacagcggaatgggttctattggaccaaactcgaaattctcttttttgggaaaataaaaccaagggagctaaccagagccaaacaccctgcgtccaagtcttagcaggcatgactatagccaccagctacctgggcatatcagcagtctcagaattttttggaacctccctcacccccttatttcatttccatatctctacatgccttaaaactcaaggagccttttatatttgtggccagtcgattcaccaatgcctccccagtaactggactggaacttgtaccataggctatgtaaccccagacatcttcatagcccctggcaatctctctcttccaataccaatctatgggaattccccgttgcccagggtgaggagggcaatccatttcattccccttctcgcgggactcggcattctagctggtacgggaaccggaattgctggaatcacaaaagcttccctcacctatagccagctctcaaaggaaatagccaacaacattgacaccatggctaaagccttaacgaccatgcaagaacaaatcgactctttagcagccgtagtccttcaaaatcgtcgaggactagacatgttaacggcagcacagggaggaatttgtttggccttagatgaaaaatgttgcttttgggtaaatcaatcaggaaaagtacaagacaacatcagacaactcctaaatcaagcctccagtttacgggaacgagccactcagggttggttaaattgggaaggaacttggaaatggttctcttgggttcttccccttacaggcccacttgttagtctcctacttttgctcctttttggtccatgtctcctaaatctaataacccaatttgtctcctctcgccttcaggccataaagctccagacgaatctcagtgcaggacgccatcctcgcaatattcaagagtcacccttctaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttat

SEQ ID NO: 7 - DNA, 20 bp, Artificial Sequence
  synthetic oligonucleotide primer PSI forward
caggactcggcttgctgaag

SEQ ID NO: 8 - DNA, 20 bp, Artificial Sequence
  synthetic oligonucleotide primer PSI reverse
tcccccgcttaatactgacg

SEQ ID NO: 9 - DNA, 19 bp, Artificial Sequence
  synthetic oligonucleotide probe PSI (FAM)
cgcacggcaagaggcgagg

SEQ ID NO: 10 - DNA, 21 bp, Artificial Sequence
  synthetic oligonucleotide primer Titin forward
aaaacgagcagtgacgtgagc

SEQ ID NO: 11 - DNA, 20 bp, Artificial Sequence
  synthetic oligonucleotide primer Titin reverse
ttcagtcatgctgctagcgc

SEQ ID NO: 12 - DNA, 25 bp, Artificial Sequence
  synthetic oligonucleotide probe Titin (VIC)
tgcacggaagcgtctcgtctcagtc

SEQ ID NO: 13 - DNA, 20 bp, Artificial Sequence
  synthetic oligonucleotide primer WPRE forward
ggcactgacaattccgtggt

SEQ ID NO: 14 - DNA, 20 bp, Artificial Sequence
  synthetic oligonucleotide primer WPRE reverse
agggacgtagcagaaggacg

SEQ ID NO: 15 - DNA, 23 bp, Artificial Sequence
  synthetic oligonucleotide probe WPRE (FAM)
acgtcctttccatggctgctcgc

SEQ ID NO: 16 - DNA, 22 bp, artificial sequence
  synthetic oligonucleotide primer albumin forward
gctgtcatctcttgtgggctgt

SEQ ID NO: 17 - DNA, 21 bp, artificial sequence
  synthetic oligonucleotide primer Albumin reverse
actcatgggagctgctggttc

SEQ ID NO: 18 - DNA, 26 bp, artificial sequence
  synthetic oligonucleotide probe Albumin (VIC)
cctgtcatgcccacacaaatctctcc

SEQ ID NO: 19 - DNA, 19 bp, artificial sequence
  synthetic oligonucleotide primer Psi-F
agcctcaataaagcttgcc

SEQ ID NO: 20 - DNA, 19 bp, artificial sequence
  synthetic oligonucleotide primer RRE-R
tctgatcctgtcgtaaggg

SEQ ID NO: 21 - DNA, 20 bp, artificial sequence
  synthetic oligonucleotide primer mLy6e forward
cgggctttgggaatgtcaac

SEQ ID NO: 22 - DNA, 20 bp, artificial sequence
  synthetic oligonucleotide primer mLy6e Reverse
gtgggatactggcacgaagt

SEQ ID NO: 23 - DNA, 17 bp, artificial sequence
  synthetic oligonucleotide primer hLy6e forward
agacctgttccccggcc

SEQ ID NO: 24 - DNA, 19 bp, artificial sequence
  synthetic oligonuceotide primer hLy6e reverse
cagctgatgcccatggaag

SEQ ID NO: 25 - DNA, 20 bp, artificial sequence
  synthetic oligonucleotide primer PO reverse
ctccaagcagatgcagcaga

SEQ ID NO: 26 - DNA, 20 bp, artificial sequence
  synthetic oligonucleotide primer PO forward
accatgatgcgcaaggccat